(19) United States
(12) Patent Application Publication
(10) Pub. No.: US 2026/0308178 A1
(43) Pub. Date: Oct. 8, 2026

(54) NOVEL COMPOUND OR SALT THEREOF, CATALYST, METHOD OF PREPARING NOVEL COMPOUND OR SALT THEREOF AND METHOD OF PREPARING MANNICH PRODUCT

(71) Applicant: OKINAWA INSTITUTE OF SCIENCE AND TECHNOLOGY SCHOOL CORPORATION, Kunigami-gun, Okinawa (JP)

(72) Inventors: Fujie TANAKA, Kunigami-gun, Okinawa (JP); . Yuvraj, Kunigami-gun, Okinawa (JP); James OSBORNE, Kunigami-gun, Okinawa (JP)

(73) Assignee: OKINAWA INSTITUTE OF SCIENCE AND TECHNOLOGY SCHOOL CORPORATION, Kunigami-gun, Okinawa (JP)

(21) Appl. No.: 19/481,980

(22) PCT Filed: May 24, 2024
(86) PCT No.: PCT/JP2024/019308

§ 371 Date: Nov. 6, 2025

(30) Foreign Application Priority Data
May 24, 2023 (JP) 2023-085779

(51) Int. Cl. C07D 515/04; B01J 31/02; C07D 211/06; C07D 291/08

(52) U.S. Cl. CPC C07D 515/04; B01J 31/0238; C07D 211/06; C07D 291/08; B01J 2231/346

(57) ABSTRACT

Provided is a novel 1,3-diamine derivative and its applications. A compound represented by a formula (I) or a salt thereof.

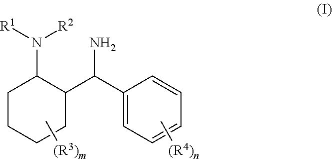

### TECHNICAL FIELD

The present disclosure relates to a novel compound or salt thereof, a catalyst, a method of preparing a novel compound or salt thereof, and a method of preparing a Mannich product.

### BACKGROUND

Amine derivatives have been used as catalysts and components of catalyst systems to accelerate chemical transformations. For example, compounds that have a primary amine group and a tertiary amine group in a geminal relationship (or 1,2-relationship), such as cinchona-derived amines, cyclohexane-1,2-diamines, and 1,2-diarylethane-1,2-diamines, for example, the following compounds:

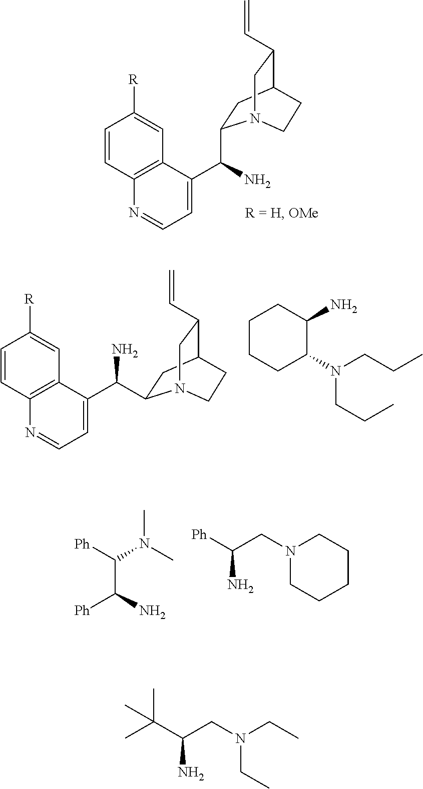

have been used as catalysts with or without other molecules (such as acids) to accelerate bond-forming reactions of enolizable ketones and aldehydes as nucleophiles.

Whereas 1,2-diamine derivatives have been extensively explored as catalysts, 1,3-diamine derivatives have not been popularly examined as catalysts. For pharmaceutical applications, propane-1,3-diamine derivatives with specific chemical structures have been proposed (PTL 1). The propane-1,3-diamine derivatives have a disubstituted amino group on the carbon to which the aryl group is attached. Other compounds with such amino group have been reported in NPTL 1.

Further, the propane-1,3-diamine derivatives having a monosubstituted amino group on the carbon to which the aryl group is attached have also been reported (NPTLs 2-4). The monosubstituted amino group has a substituent such as tosyl group, and methane sulfonate group.

### CITATION LIST

### Patent Literature

PTL 1: JP2005-50330A

### Non Patent Literature

NPTL 1: B Meria, et al. “A simple and Highly Diastereoselective one-Pot Synthesis of 1,3-Diamines”, Synlett 1997, p. 177-178
NPTL 2: T. Kashiwagi, et al., “Diastereoselective synthesis of 1,3-diamines by a domino reaction of imines, enamines, and trichlorosilane”, Tetrahedron Lett. 2014, 55, p. 1924-1926.
NPTL. 3: M. Ito, et al. “Imino ene-type reaction of enamines with N-sulfonylimines and application to diastereoselective synthesis of N-sulfonyl-1,3-diamines”, Tetrahedron Lett. 2014, 55, p. 4082-4085.
NPTL. 4: M. Sugiura, et al. “Stereoselective Synthesis of Nitrogen-Containing Compounds from Enamines”, J. Org. Chem. 2017, 82, pp. 10968-10979.

### SUMMARY

### Technical Problem

A novel 1,3-diamine derivative and its application are expected to be developed.

### Solution to Problem

An object of this disclosure is to provide a novel 1,3-diamine derivative and its applications.

The gist of the present disclosure is as follows:
[1]A compound represented by a formula (I) or a salt thereof

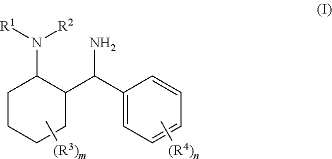

wherein
R¹ and R² are independently selected from the group consisting of an alkyl, an alkenyl, an alkynyl, a heteroalkyl, a heteroalkenyl, or a heteroalkynyl, which may be substituted with one or more selected from the group consisting of an aryl and a heteroaryl, or
R¹ and R² together form —(CH₂)ₚ—, and the —(CH₂)ₚ— forms a ring in combination with the nitrogen atom that the R¹ and the R² are both attached to,
wherein p is an integer from 2 to 8, and
the —(CH₂)ₚ— may be interrupted by one to three hetero atom(s) and may be substituted with one or more selected from the group consisting of an alkyl, an aryl, and a heteroaryl;
R³ is independently selected from the group consisting of an alkyl, and an aryl, or two adjacent R³s may form a ring with carbon atoms they are attached to;
R⁴ is independently selected from the group consisting of a halogen atom, an alkyl, an alkoxy, a cyano, a nitro, and an acylamido, or
two adjacent R⁴s may form a ring in combination with carbon atoms they are attached to;
m is an integer from 0 to 8; and
n is an integer from 0 to 5.
[2] The compound or the salt thereof according to [1], wherein the compound is represented by formula (1-1) or (1-2)

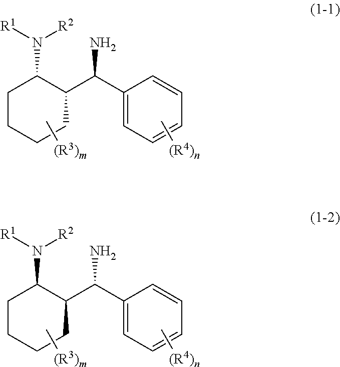

wherein R¹, R², R³, R⁴, m, and n are as defined in [1].
[3]A catalyst comprising a compound or a salt thereof according to [1] or [2].
[4] The catalyst according to [3] wherein it is used in a Mannich reaction.
[5]A method of preparing a compound or a salt thereof according to [1] comprising: converting an —NHPMP moiety of a compound represented by a formula (II) to an amino group; and introducing the amino group by reductive amination of a carbonyl group of the compound represented by the formula (II), followed by converting the introduced amino group to a tertiary amino group to form an —NR¹R² moiety, wherein R¹, and R² are as defined in [1].

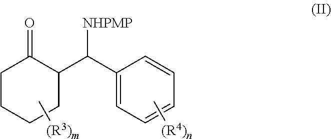

wherein
PMP is a p-methoxyphenyl group, and
R³, R⁴, m, and n are as defined in [1].
[6]A method of preparing a Mannich product, comprising:
reacting a compound represented by formula (VIII) with a compound represented by formula (VII), in the presence of a compound represented by the formula (I) according to [1]

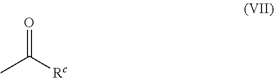

wherein
R^{c} is a hydrogen atom or a monovalent hydrocarbon group which may be substituted with a —P(═O)(OR^{d})(OR^{e}), a —C(═O)R^{d}, a —S(═O)₂R^{d}, a —C(═O)OR^{d}, or a —C(═O)NR^{f}R^{g},
wherein
R^{d} is an alkyl,
R^{e} is an alkyl,
R^{f} is a hydrogen atom or an alkyl, and
R^{g} is a hydrogen atom or an alkyl;

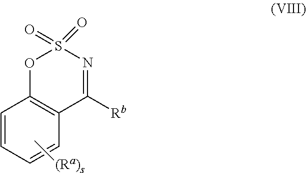

wherein
R^{a} is independently selected from the group consisting of an alkyl, a halogen atom, an alkoxy, and a nitro, or two adjacent R^{a}s may form a ring in combination with carbon atoms they are attached to;
s is an integer of 0 to 4; and
R^{b} is a hydrogen atom or an alkoxycarbonyl.
[7] The method of preparing a Mannich product according to [6], comprising: reacting the compound represented by the formula (VIII) with the compound represented by the formula (VII), in the presence of the compound represented by the formula (1-1) according to [2] to obtain a compound represented by formula (9-1), or in the presence of the compound represented by the formula (1-2) according to [2] to obtain a compound represented by formula (9-2)

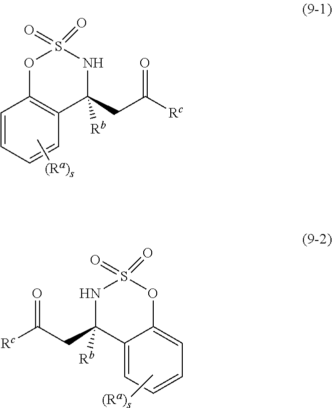

wherein R^{a}, R^{b}, R^{c} and s are as defined in [6].
[8] The method of preparing a Mannich product according to [6], comprising: reacting the compound represented by the formula (VIII) with the compound represented by the formula (VII′) to obtain a compound represented by formula (9-3)

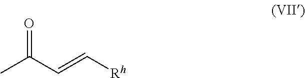

wherein R^{h} is a hydrogen atom, a —P(═O)(OR^{d})(OR^{e}), a —C(═O)R^{d}, a —S(═O)₂R^{d}, a —C(═O)OR^{d}, a —C(═O)NR^{f}R^{g}, or a monovalent hydrocarbon group which may be substituted with a —P(═O)(OR^{d})(OR^{e}), a —C(═O)R^{d}, a —S(═O)₂R^{d}, a —C(═O)OR^{d}, or a —C(═O)NR^{f}R^{g},
wherein R^{d}, R^{e}, R^{f}, and R^{g} are as defined in [6];

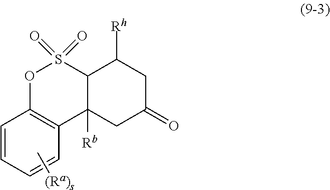

wherein
R^{a}, R^{b}, and s are as defined in [6]; and
R^{h} is as defined in the above.

### Advantageous Effect

According to the present disclosure, novel 1,3-diamine derivatives and their applications, including catalytic uses, are provided.

### BRIEF DESCRIPTION OF DRAWINGS

In the accompanying drawings:

FIG. 1: The X-ray crystal structure of compound 1a·CF₃COOH.

FIG. 2: The X-ray crystal structure of compound 1g·CF₃COOH.

FIG. 3: The X-ray crystal structure of compound 9g.

### DETAILED DESCRIPTION

The presently disclosed techniques will be described in detail below.

### Terms

### (Monovalent Hydrocarbon Group)

The term “monovalent hydrocarbon group” refers to a monovalent group consisting of carbon atom(s) and hydrogen(s). Examples of the monovalent hydrocarbon group include, without limitation, alkyl, alkenyl, alkynyl, cycloalkyl, cycloalkenyl, cycloalkynyl, aryl, aralkyl.

### (Alkyl)

The term “alkyl” refers to a straight-chain or branched saturated hydrocarbon group. The alkyl preferably has from 1 to 12 carbon atoms (C₁₋₁₂ alkyl), more preferably from 1 to 8 carbon atoms (C₁₋₈ alkyl). Examples of the alkyl include, without limitation, methyl, ethyl, propyl (e.g., n-propyl, isopropyl), butyl (e.g., n-butyl, tert-butyl, secbutyl, iso-butyl), pentyl (e.g., n-pentyl, 3-pentanyl, amyl, neopentyl, 3-methyl-2-butanyl, tertiary amyl), and hexyl (e.g., n-hexyl, ethylhexyl), heptyl (e.g., n-heptyl), octyl (e.g., n-octyl), nonyl (e.g., n-nonyl), decyl (e.g., n-decyl), undecyl (e.g., n-undecyl), and dodecyl (e.g., n-dodecyl). Among the above, the alkyl is preferably methyl, ethyl, or propyl.

### (Alkenyl)

The term “alkenyl” refers to a straight-chain or branched unsaturated hydrocarbon group having one or more carbon-carbon double bonds (e.g., 1, 2, 3, or 4 double bonds). The one or more carbon-carbon double bonds can be internal (such as in 2-butenyl) or terminal (such as in 1-butenyl). The alkenyl preferably has from 2 to 12 carbon atoms (C₂₋₁₂ alkenyl), more preferably from 2 to 8 carbon atoms (C₂₋₈ alkenyl). Examples of the alkenyl include, without limitation, ethenyl, 1-propenyl, 2-propenyl, 1-butenyl, 2-butenyl, butadienyl, pentenyl, pentadienyl, hexenyl, heptenyl, octenyl, octatrienyl, nonenyl, and decenyl. Among the above, the alkenyl is preferably ethenyl, 1-propenyl, or 1-butenyl.

### (Alkynyl)

The term “alkynyl” refers to a straight-chain or branched unsaturated hydrocarbon group having one or more carbon-carbon triple bonds (e.g., 1, 2, 3, or 4 triple bonds) (“C₂₋₁₀ alkynyl”). The one or more carbon-carbon triple bonds can be internal (such as in 2-butynyl) or terminal (such as in 1-butynyl). The alkynyl preferably has from 2 to 12 carbon atoms (C₂₋₁₂ alkynyl), more preferably from 1 to 8 carbon atoms (C₁₋₈ alkynyl). Examples of the alkynyl include, without limitation, ethynyl, 1-propynyl, 2-propynyl, 1-butynyl, 2-butynyl, pentynyl, hexynyl, pentynyl, octynyl, nonynyl, and decynyl. Among the above, the alkynyl is preferably ethynyl, 1-propynyl, or 1-butynyl.

### (Heteroalkyl)

The term “heteroalkyl” refers to an above alkyl group, which further includes at least one heteroatom (e.g., 1, 2, 3, or 4 heteroatoms) selected from oxygen, nitrogen, sulfur within (i.e., inserted between adjacent carbon atoms of) and/or placed at one or more terminal position(s) of the parent chain.

### (Heteroalkenyl)

The term “heteroalkenyl” refers to an above alkenyl group, which further includes at least one heteroatom (e.g., 1, 2, 3, or 4 heteroatoms) selected from oxygen, nitrogen, sulfur within (i.e., inserted between adjacent carbon atoms of) and/or placed at one or more terminal position(s) of the parent chain.

### (Heteroalkynyl)

The term “heteroalkynyl” refers to an above alkynyl group, which further includes at least one heteroatom (e.g., 1, 2, 3, or 4 heteroatoms) selected from oxygen, nitrogen, sulfur within (i.e., inserted between adjacent carbon atoms of) and/or placed at one or more terminal position(s) of the parent chain.

### (Aryl)

The term “aryl” refers to a monocyclic or polycyclic group (e.g., bicyclic or tricyclic) having zero heteroatoms provided in the aromatic ring system. The aryl preferably has 6-20 ring carbon atoms in the aromatic ring system (“C₆₋₂₀ aryl”), and more preferably 6-14 ring carbon atoms in the aromatic ring system (“C₆₁₄ aryl”). Examples of the aryl include phenyl, naphthyl such as 1-naphthyl and 2-naphthyl, and anthracyl.

### (Heteroaryl)

The term “heteroaryl” refers to a monocyclic or polycyclic group (e.g., bicyclic or tricyclic) having 1, 2, 3, or 4 heteroatoms selected from oxygen, nitrogen, sulfur, the remaining ring atoms being carbon provided in the aromatic ring system. The heteroaryl preferably has 5-20 ring atoms, and more preferably 5-14 ring atoms. Examples of the heteroaryl include, without limitation, furyl, thienyl, pyrrolyl, imidazolyl, oxazolyl, thiazolyl, isoxazolyl, pyrazolyl, pyridyl, pyridazinyl, indolyl, quinolyl, and isoquinolyl.

### (Alkoxy)

The term “alkoxy” is represented by “R—O— (wherein R is the above alkyl)”. Examples of the alkoxy include, without limitation, methoxy, ethoxy, propoxy, 2-propoxy, butoxy, and tert-butoxy.

### (Halogen)

The term “halogen” refers to fluorine (fluoro, —F), chlorine (chloro, —Cl), bromine (bromo, —Br), or iodine (iodo, —I).

### <1,3-Diamine Derivative>

The present disclosure is related to a novel 1,3-amine derivative which is a compound represented by a formula (I) and a salt thereof.

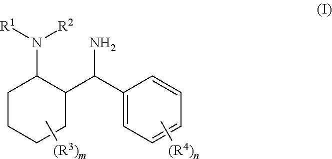

R¹ and R² may be independently an alkyl, an alkenyl, an alkynyl, a heteroalkyl, a heteroalkenyl, or a heteroalkynyl, preferably, a C₁₋₈ alkyl (e.g., a methyl, an ethyl).

R¹ and R² may together form —(CH₂)ₚ—, and the —(CH₂)ₚ— forms a ring in combination with the nitrogen atom that the R¹ and the R² are both attached to, wherein p is an integer from 2 to 8, preferably an integer from 4 to 8.

The —(CH₂)ₚ— may be interrupted by one to three hetero atom(s) and may be substituted with one or more selected from the group consisting of an alkyl, an aryl, and a heteroaryl.

Preferably, R¹ and R² together form —(CH₂)ₚ—, wherein p is an integer from 2 to 8, the —(CH₂)ₚ— is uninterrupted or interrupted by one or two oxygen atom(s), one or two sulfur atom(s), or one or two nitrogen atom(s). Preferably it is interrupted by one oxygen atom.

Preferred examples of the rings formed by R¹ and R² in combination with the nitrogen atom include pyrrolidine, piperidine, azepazine, piperidine, morpholine, and thiomorpholine.

The subscript “m” is an integer from 0 to 8, preferably an integer from 0 to 4, more preferably from 0 to 2, and further more preferably 0.

In case where m is an integer from 1 to 8, R³ may be an alkyl, or an aryl, and preferably a C₁₋₈ alkyl (e.g., a methyl, an ethyl), and a phenyl.

In the case where m is an integer from 2 to 8, two adjacent R³s may form a ring with the carbon atoms they are attached to. The ring formed by two adjacent R³s in combination with the carbon atoms may be an aliphatic ring. The number of atoms constituting the ring may be 6 to 20. Preferred examples of the rings include a cyclohexane.

The subscript “n” is an integer from 0 to 5, preferably an integer from 0 to 2, and more preferably 0 or 1.

In case where n is an integer from 1 to 5, R⁴ may be a halogen atom, an alkyl, an alkoxy, a cyano, a nitro, and an acylamido, preferably a halogen atom, more preferably a bromine atom.

In case where n is an integer from 2 to 4, two adjacent R⁴s may form a ring with carbon atoms they are attached to. The ring formed by two adjacent R⁴s in combination with carbon atoms may be an aromatic ring. The number of atoms constituting the ring may be 6 to 20. Preferred examples of the ring include benzene, and piperidine.

Preferred examples of the compound (I) include a compound represented by a formula (I′).

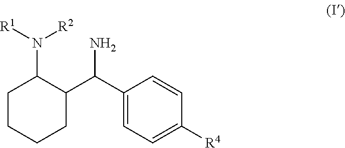

wherein the definitions and examples of R¹, R², and R⁴ are as mentioned above.

The compound (I) may be in a form of a salt. The salt may be an acid addition salt of an inorganic acid or an organic acid. Examples of the inorganic acid and the organic acid include hydrochloric acid, nitric acid, sulfuric acid, phosphoric acid, citric acid, formic acid, fumaric acid, maleic acid, acetic acid, trifluoroacetic acid, succinic acid, tartaric acid, methane sulfonic acid, and p-toluenesulfonic acid.

The compound (I) encompasses a racemic mixture, diastereomers, and enantiomers. The following compounds represented by formula (1-1) to (1-4) may be mentioned for the diastereomers, and enantiomers.

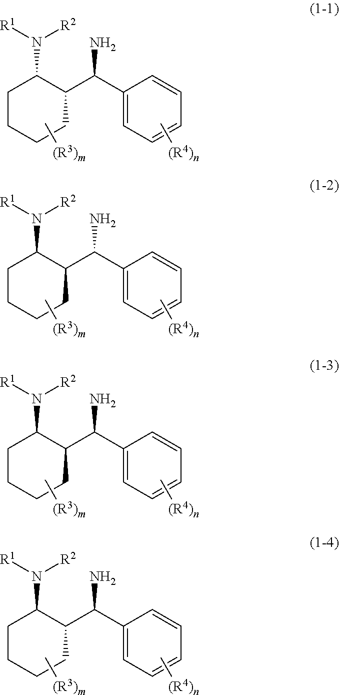

wherein the definitions and examples of R¹, R², R³, R⁴, m, and n are as mentioned above.

### <Method of Preparing the Compound (I)>

The compound (I) may be prepared as follows:
converting an —NHPMP moiety of a compound represented by a formula (II) to an amino group; and
introducing the amino group by reductive amination of a carbonyl group of the compound represented by the formula (II), followed by converting the introduced amino group to a tertiary amino group to form an —NR¹R² moiety, wherein the definition and examples of R¹, and R² are as mentioned above.

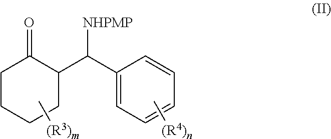

wherein
PMP is a p-methoxyphenyl group, and
the definition and examples of R³, R⁴, m, and n are as mentioned above.

The compound (II) may be prepared by the method described in the following literature:
Y. Garg, F. Tanaka, Org. Lett. 2020, 22, 4542.

The conversion of the −NHPMP moiety of the compound (II) to the amino group may be performed by using a deprotection agent such as an ammonium cerium nitrate (CAN). For example, the reaction may be performed by using CAN in an appropriate solvent (e.g., a mixture of CH₃CN and H₂O) at low temperature (e.g., −10° C. to 10° C.). A deprotection agent other than CAN may be used for the conversion. The converted amino group may be protected with Boc group.

The introduction of the amino group by reductive amination of a carbonyl group of the compound (II) may be performed by using a reducing agent such as Na(OAc)₃BH, and NaBH₃CN. For example, the compound (II) may be reacted with p-anisidine by using Na(OAc)₃BH, and CH₃COOH in an appropriate solvent (e.g., 1,2-dichloroethane) at low temperature (e.g., −10° C. to 10° C.). The introduced PMP group may be deprotected by using a deprotection agent mentioned above. For example, the deprotection may be performed by using CAN in the mixture of CH₃CN and H₂O at 0° C.

The conversion of the introduced amino group to a tertiary amino group to form an —NR¹R² moiety may be performed using an appropriate reagent depending on R¹ and R². The reagent may be a halide (e.g., bromide) containing moiety corresponding to R¹ and R². For example, when R¹ and R² together form a ring, the reagent may be 1,5-dibromopentane, 1,4-dibromobutane, 1,6-dibromohexane, or bis(2-bromoethyl)ether.

The reaction may be performed in the presence of a base. Examples of the base include K₂CO₃. The base may be used alone or in a combination of two or more bases. The reaction may be performed in an appropriate solvent (e.g., CH₃CN) at 25° C. to 60° C. The solvent may be used alone or in a combination of two or more solvents.

The Boc group may be deprotected by trifluoroacetic acid. The obtained reaction product may be subjected to purification,

For example, a salt of compound (1a) can be prepared by the following method using the compound (2). The compound (2) can be generated by the (S)-[-proline-catalyzed anti-selective Mannich reaction of cyclohexanone with an aryl imine derivative in the presence of K₂CO₃ (Scheme 1). The compound (1a) can be prepared via the route of the compounds from (3) to (6) as illustrated in Scheme 1. Depending on workup conditions after the deprotection of Boc group, the compound (1a) is obtained in the CF₃COOH salt form in Scheme 1.

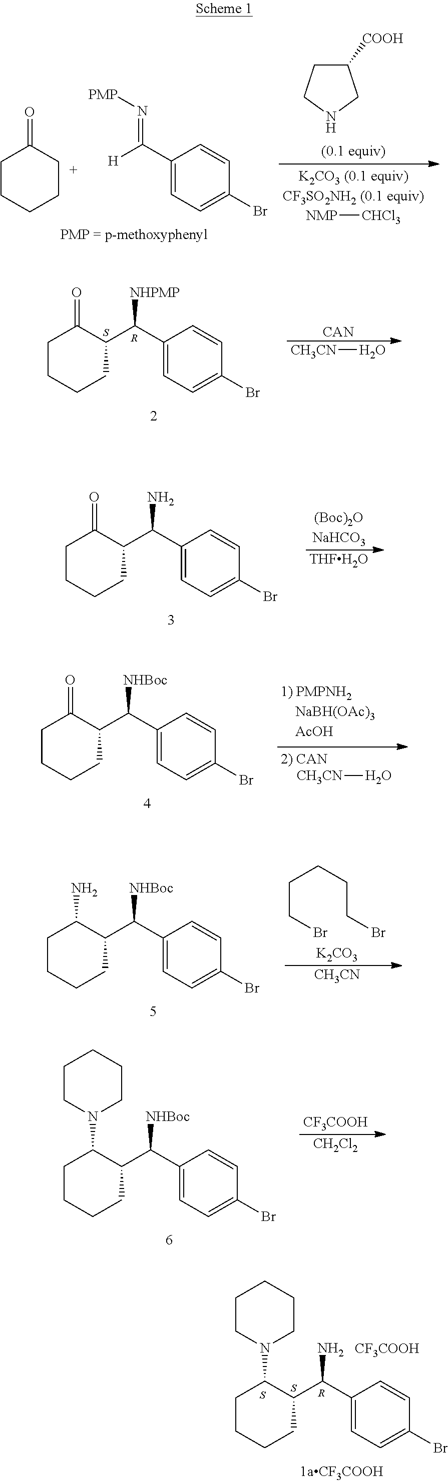

The compound (1a) in the free form can be prepared by changing the workup conditions after the deprotection of Boc group, for example, as illustrated in Scheme 2.

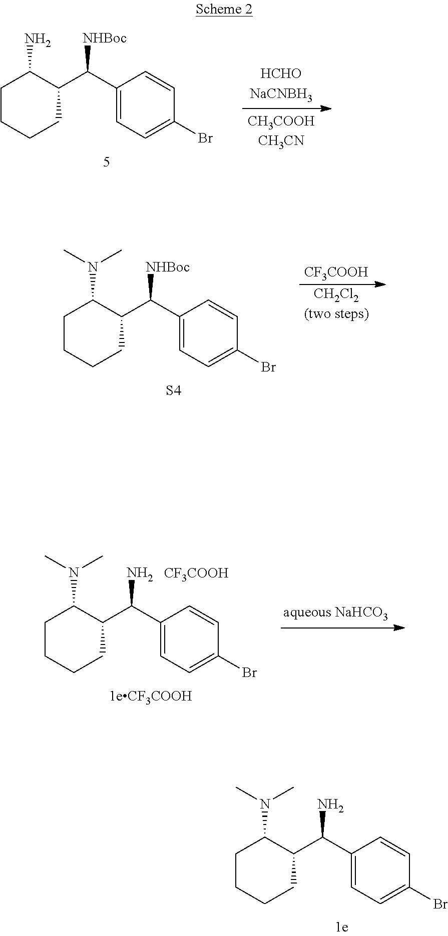

Other compounds included in the compound (I) and their salts can also be prepared by methods similar to those shown in Schemes 1 and 2, using appropriate starting substances, and reaction conditions.

### <Catalyst>

The compound (I) or a salt thereof can be used as a catalyst in various reactions. The present disclosure is also related to a catalyst comprising a compound (I) or the salt. The compound (I) or a salt thereof may be used as a catalyst in a reaction for preparing a Mannich product. In particular, the compound (I) or a salt thereof is useful as a catalyst in a Mannich reaction including a Mannich-aza-Michael reaction. These reactions are advantageous for synthesizing the Mannich product.

A catalyst comprising the compound (I) or a salt thereof may be used in combination with an acid. Examples of the acid include trifluoroacetic acid. The acid may be used alone or in a combination of two or more acids. The catalyst may be used in combination with dibenzyl phosphate. It is preferred that the catalyst used in combination with trifluoroacetic acid and dibenzyl phosphate.

An example of the Mannich reaction in which the compound (I) or a salt thereof can be used as a catalyst is as follows:
reacting a compound represented by a formula (VIII) with a compound represented by a formula (VII)

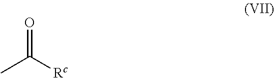

wherein
R^{c} is a hydrogen atom or a monovalent hydrocarbon group which may be substituted with a —P(═O)(OR^{d})(OR^{e}), a —C(═O)R^{d}, a —S(═O)₂R^{d}, a —C(═O)OR^{d}, or a —C(═O)NR^{f}R^{g}, wherein
R^{d} is an alkyl,
R^{e} is an alkyl,
R^{f} is a hydrogen atom or an alkyl, and
R^{g} is a hydrogen atom or an alkyl.
R^{c} is preferably, an alkyl, an alkenyl, an aryl, a —CH₂—P(═O)(OR^{d})(OR^{e}), a —CH₂—C(═O)R^{d}, a —CH₂—S(═O)₂R^{d}, a —CH₂—C(═O)OR^{d}, or a —CH₂—C(═O)NR^{f}R^{g}, wherein R^{d}, R^{e}R^{f}, and R^{g} are as defined in the above.

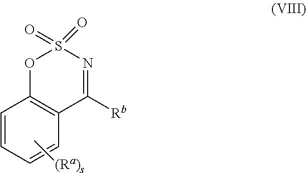

wherein
R^{d} is independently selected from the group consisting of an alkyl, a halogen atom, an alkoxy and a nitro, or
two adjacent R^{a}s may form a ring in combination with the carbon atoms they are attached to;
s is an integer of 0 to 4; and
R^{b} is a hydrogen atom or an alkoxycarbonyl.

A Mannich product represented by a formula (IX) may be mentioned as a reaction product.

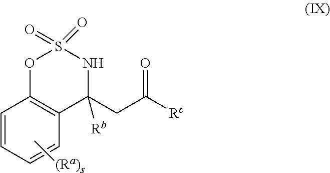

wherein R^{a}, R^{b}, R^{c}, and s are as defined above.

In the reaction, an amount of the compound (VII) may be 1.5 to 5.0 mol, preferably 2.0 to 4.0 mol, relative to 1.0 mol of the compound (VIII).

The compound (I) may be used in an amount of 0.1 to 0.8 mol, preferably 0.15 to 0.50 mol, relative to 1.0 mol of the compound (VII).

The compound (I) is preferably used with an acid(s) such as trifluoroacetic acid and/or dibenzyl phosphate in a catalytic system. The acid(s) may be used in an amount of 0.1 to 0.8 mol, preferably 0.15 to 0.50 mol, relative to 1.0 mol of the compound (VII). The dibenzyl phosphate may be used in an amount of 0.1 to 0.8 mol, preferably 0.15 to 0.50 mol, relative to 1.0 mol of the compound (VII).

The reaction may be performed in a solvent. Examples of the solvent include an aromatic hydrocarbon solvent such as benzene, toluene, xylene). The reaction may be performed in the temperature range of −10 to 60° C., preferably performed at room temperature (25° C.).

In particular, the enantiomers of the compound (1-1) or the compound (1-2) mentioned above accelerate the enantioselective, γ-position-selective Mannich reactions of β-ketophosphonates. They also accelerate the enantioselective γ-position-selective Mannich reactions of β-ketocarbonyl compounds.

As illustrated in Scheme 3, the compound (1a) which is included in the compound (1-1) has a primary amine group and a tertiary amine group with a 1,3-relationship (Scheme 1). The compound (1a) accelerates enantioselective, γ-position-selective Mannich reactions of Mannich products.

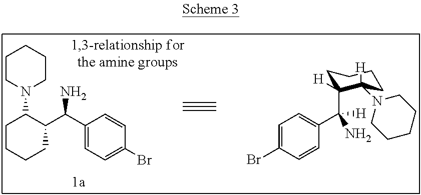

With respect to the compounds (1-1) and (1-2), the primary amine group may lead to the formation of enamine intermediates during the catalysis. It is presumed that the attachment of the tertiary amine group and the amino(aryl)methyl group moiety on a cyclohexane ring to position the amine groups in a 1,3-relationship would result in catalytic functions.

In case where the above Mannich reaction is performed in the presence of the compound (1-1), the compound represented by the formula (9-1) may be obtained, and in the presence of the compound (1-2), the compound represented by the formula (9-2) may be obtained,

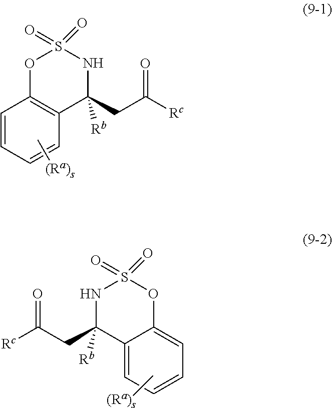

wherein R^{a}, R^{b}, R^{c}, and s are as defined above.

In order to obtain the compound (9-1) or the compound (9-2) with high yield with high enantioselectivities, it is preferred that the reaction is carried out by using acid addition salt of the compound (1) as a catalyst together with dibenzyl phosphate. It is also preferred that the reaction is carried out by the compound (1) in a free form as a catalyst together with an acid (e.g. a trifluoroacetic acid) and dibenzyl phosphate.

In the case where a compound represented by a formula (VII′) is used as the compound of the formula (VII), a Mannich-aza-Michael reaction takes place and a cyclized Mannich product represented by a formula (9-3) may be obtained.

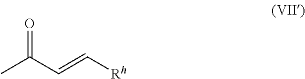

wherein R^{h} is a hydrogen atom, a —P(═O)(OR^{d})(OR^{e}), a —C(═O)R^{d}, a —S(═O)₂R^{d}, a —C(═O)OR^{d}, a —C(═O)NR^{f}R^{g}, or a monovalent hydrocarbon group which may be substituted with a —P(═O)(OR^{d})(OR^{e}), a —C(═O)R^{d}, a —S(═O)₂R^{d}, a —C(═O)OR^{d}, or a —C(═O)NR^{f}R^{g}, wherein R^{d}, R^{e}, R^{f}, and R^{g} are as defined in the above;

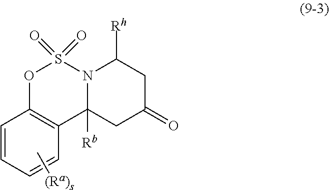

wherein
R^{a}, R^{b}, and s are as defined in the above; and
R^{h} is as defined in the above.

R^{h} is preferably a hydrogen atom, analkyl, anaryl, a —CH₂—P(═O)(OR^{d})(OR^{e}), a —CH₂—C(═O)R^{d}, a —CH₂—S(═O)₂R^{d}, a —CH₂—C(═O)OR^{d}, or a —CH₂—C(═O)NR^{f}R^{g} (wherein R^{d}, R^{e}, R^{f}, and R^{g} are as defined in the above), more preferably, a hydrogen atom or a straight-chain alkyl. The straight-chain alkyl preferably has 1 to 12 carbon atoms, more preferably 1 to 8 carbon atoms, further more preferably 1 to 3 carbon atoms.

In this reaction, R^{b} is preferably a hydrogen.

In the reaction, an amount of the compound (VII′) may be 1.5 to 5.0 mol, preferably 2.0 to 4.0 mol, relative to 1.0 mol of the compound (VIII).

The compound (I) may be used in an amount of 0.1 to 0.8 mol, preferably 0.15 to 0.50 mol, relative to 1.0 mol of the compound (VII′).

The compound (I) is preferably used with an acid(s) such as trifluoroacetic acid and/or dibenzyl phosphate in a catalytic system. The acid(s) may be used in an amount of 0.1 to 0.8 mol, preferably 0.15 to 0.50 mol, relative to 1.0 mol of the compound (VII′). The dibenzyl phosphate may be used in an amount of 0.1 to 0.8 mol, preferably 0.15 to 0.50 mol, relative to 1.0 mol of the compound (VII′).

The reaction may be performed in a solvent. Examples of the solvent include an aromatic hydrocarbon solvent such as benzene, toluene, xylene). The reaction may be performed in the temperature range of −10 to 60° C., preferably at room temperature (25° C.).

### Application

Applications of the compound (I) or a salt thereof are not limited to a catalyst, but include pharmaceuticals, raw materials for polymeric materials.

### EXAMPLES

The following provides more specific descriptions of the present disclosure based on certain examples.

### Example 1 (Synthesis of Catalysts)

### 1. Synthesis of Compound 1a

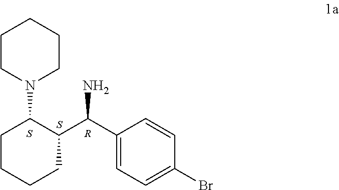

Compound 1a was synthesized as illustrated in Scheme 4.

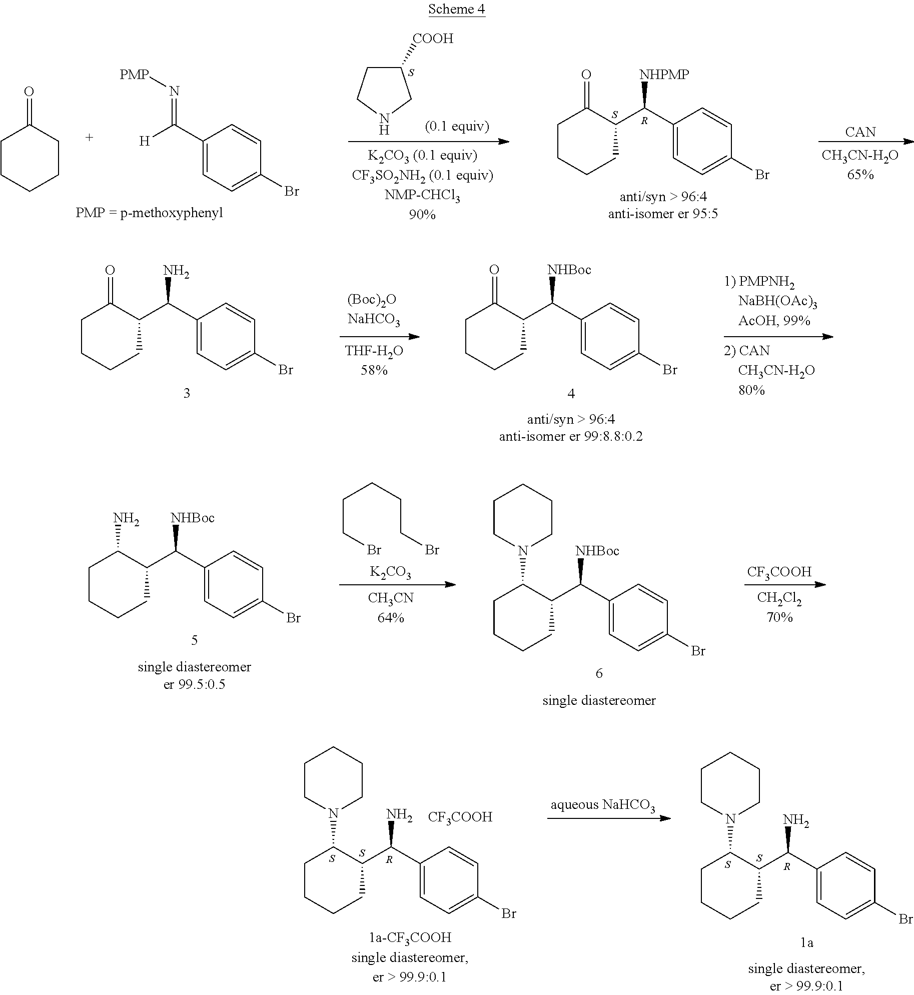

### (Synthesis of Compound 2)

Compound 2 was synthesized by the procedures disclosed in Y. Garg, F. Tanaka, Org. Lett. 2020, 22, 4542.

K₂CO₃ (691 mg, 5.00 mmol) was added to a solution of (S)-3-pyrrolidinecarboxylic acid (575 mg, 5.00 mmol) in N-methylpyrrolidone (NMP) (15 mL), and the mixture was stirred at room temperature (25° C., rt) for 1 h. The mixture was cooled to 0° C., and CF₃SO₂NH₂ (745 mg, 5.00 mmol), CHCl₃ (35 mL), and cyclohexanone (51.8 mL, 500 mmol) were added to the mixture. The mixture was stirred at the same temperature for 5 min, and the N-p-methoxyphenyl (PMP)-protected imine (14.5 g, 50.0 mmol), which was prepared from 4-bromobenzaldehyde and p-anisidine, was added. The mixture was stirred at 0° C. for 192 h. The mixture was diluted with EtOAc (100 mL) and then H₂O (100 mL) was added to the mixture, and the mixture was extracted with EtOAc. Organic layers were combined, washed with brine, dried over anhydrous Na₂SO₄, filtered, concentrated in vacuo, and purified by flash column chromatography (hexane/EtOAc=85:15) to give Compound 2 (17.4 g, 90%, anti/syn >96:4, er 95:5) as a colorless oil.

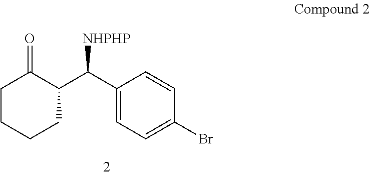

The characterization data of Compound 2 was consistent with the data described in the literature Organic Letters 2020, 22, 4542.

### (Synthesis of Compound 3)

A solution of ammonium cerium nitrate (CAN) (120 g, 220 mmol) in H₂O (300 mL) was added to a solution of Compound 2 (anti/syn >96:4, er 95:5, 19.40 g, 50.0 mmol) in CH₃CN (400 mL) at 0° C., and the mixture was stirred at the same temperature for 2 h. The mixture was diluted with water (500 mL) and extracted with EtOAc (100 mL×3). Organic layers were combined, dried over Na₂SO₄, filtered, concentrated, and purified by flash column chromatography (CH₂Cl₂/MeOH=90:10) to give Compound 3 (8.70 g, 63%, dr>96:4) as a colorless oil.

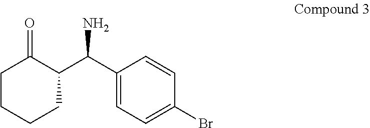 [SMILES: N[C@@H](c1ccc(Br)cc1)[C@@H]1CCCCC1=O]

¹H NMR (500 MHz, CD₃OD): δ 7.64 (d, J=8.5 Hz, 2H), 7.35 (d, J=8.5 Hz, 2H), 4.44 (d, J=10.0 Hz, 1H), 3.11-3.04 (m, 1H), 2.58-2.43 (m, 2H), 2.18-2.07 (m, 1H), 1.84-1.75 (m, 1H), 1.75-1.54 (m, 3H), 1.28 (qd, J=13.0 Hz, 4.0 Hz, 1H). ¹³C NMR (125 MHz, CD₃OD): δ 213.1, 135.7, 133.6, 130.9, 124.5, 56.1, 53.6, 43.0, 32.7, 28.9, 25.5. HRMS (ESI): m/z calcd for C₁₃H₁₇NOBr [M+H]⁺ 282.0488, found 282.0497.

### (Synthesis of Compound 4)

NaHCO₃ (7.77 g, 92.5 mmol) and di-tert-butyl decarbonate (8.5 mL, 37 mmol) were added to a solution of Compound 3 (dr>96:4, 8.70 g, 30.9 mmol) in THF/H₂O (1:1 (v/v), 100 mL) at 0° C., and the mixture was stirred at room temperature (25° C.) for 12 h. The mixture was concentrated to remove the organic solvent and extracted with EtOAc (50 mL×3). Organic layers were combined, dried over Na₂SO₄, concentrated, and crystallized from EtOH to give Compound 4 (6.84 g, 58%, anti/syn >96:4, anti-isomer er 99.8:0.2) as a colorless solid.

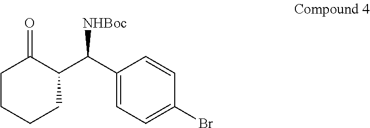 [SMILES: CC(C)(C)OC(=O)N[C@@H](c1ccc(Br)cc1)[C@@H]1CCCCC1=O]

¹H NMR (500 MHz, CDCl₃): δ 7.41 (d, J=8.5 Hz, 2H), 7.18 (d, J=8.5 Hz, 2H), 5.79 (brs, 1H), 4.82-4.72 (m, 1H), 2.87-2.80 (m, 1H), 2.38-2.32 (m, 1H), 2.32-2.23 (m, 1H), 2.16-2.07 (m, 1H), 2.07-1.97 (m, 1H), 1.94-1.85 (m, 1H), 1.78-1.64 (m, 3H), 1.40 (s, 9H). ¹³C NMR (125 MHz, CDCl₃): δ 212.5, 155.6, 140.8, 131.3, 128.3, 120.7, 79.6, 55.9, 54.8, 42.6, 32.5, 28.3, 28.0, 24.4. HRMS (ESI): m/z calcd for C₁₋₈ H₂₅NO₃Br [M+H]⁺ 382.1012, found 382.1017. HPLC (Daicel Chiralpak IC-3, hexane/i-PrOH=90:10, flow rate=0.5 mL/min, λ=220 nm): t_{R} 17.7 min (minor enantiomer), 42.5 min (major enantiomer). For (±)-4 (anti/syn=5:1), HPLC (Daicel Chiralpak IC-3, hexane/i-PrOH=90:10, flow rate=0.5 mL/min, λ=220 nm): t_{R} 17.8 min (anti-isomer), 18.4 min (syn-isomer), 24.2 min (syn-isomer), 42.8 (anti-isomer).

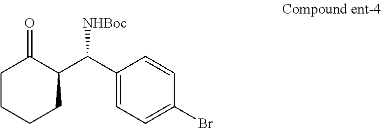 [SMILES: CC(C)(C)OC(=O)N[C@H](c1ccc(Br)cc1)[C@H]1CCCCC1=O]

Colorless solid, dr>96:4, er 0.1:>99.9. HPLC (Daicel Chiralpak IC-3, hexane/i-PrOH=90:10, flow rate=0.5 mL/min, λ=220 nm): t_{R} 16.5 min (major enantiomer). For (±)-4 (anti/syn=9:1), HPLC (Daicel Chiralpak IC-3, hexane/i-PrOH=90:10, flow rate=0.5 mL/min, λ=220 nm): t_{R} 16.5 min (anti-isomer), 17.8 min (syn-isomer), 20.8 min (syn-isomer), 32.8 (anti-isomer).

### (Synthesis of Compound 5)

p-Anisidine (2.62 g, 21.3 mmol) was added to a solution of Compound 4 (dr>96:4, er 99.8:0.2, 6.80 g, 17.8 mmol) in 1,2-dichloroethane (190 mL) at 0° C., and the mixture was stirred for 5 min. To the mixture, Na(OAc)₃BH (11.3 g, 53.4 mmol) and CH₃COOH (3.05 mL, 53.4 mmol) were added at the same temperature, and the mixture was stirred for 24 h at the same temperature. Further Na(OAc)₃BH (11.3 g, 53.4 mmol) and CH₃COOH (3.05 mL, 53.4 mmol) were added, and the mixture was stirred at the same temperature for another 24 h. The mixture was quenched by addition of saturated aqueous NaHCO₃ solution and extracted with CH₂Cl₂ (100 mL×3). The organic layers were combined, dried over Na₂SO₄, filtered, concentrated, and purified by flash column chromatography (hexane/EtOAc=90:10) to give reductive amination product (8.70 g, 99%, dr 3.4:1) as a colorless oil.

A solution of ammonium cerium nitrate (CAN) (35.5 g, 64.7 mmol) in H₂O (170 mL) was added to a solution of this compound (dr 3.4:1, 7.20 g, 14.7 mmol) in CH₃CN (230 mL) at 0° C., and the mixture was stirred at the same temperature for 2 h. The mixture was diluted with water (300 mL) and extracted with EtOAc (100 mL×3). Organic layers were combined, dried over Na₂SO₄, filtered, concentrated, and purified by flash column chromatography (CH₂Cl₂/MeOH=97:3) to give Compound 5 (4.51 g, 80%, single diastereomer, er 99.5:0.5) as a colorless oil and a mixture of 5 and its diastereomer (600 mg, 14%, dr 3:1, the major was Compound 5 and the minor was a diastereomer of Compound 5).

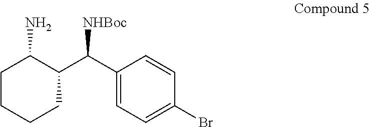 [SMILES: CC(C)(C)OC(=O)N[C@@H](c1ccc(Br)cc1)[C@@H]1CCCC[C@@H]1N]

[α]²⁵_{D}+58.0 (c 0.4, MeOH, single diastereomer, er 99.5:0.5). ¹H NMR (500 MHz, CD₃OD): δ 7.52 (d, J=8.5 Hz, 2H), 7.23 (d, J=8.5 Hz, 2H), 4.29 (d, J=11.5 Hz, 1H), 3.74-3.70 (m, 1H), 2.09-1.98 (m, 2H), 1.81-1.60 (m, 3H), 1.49-1.39 (m, 1H), 1.44 (s, 9H), 1.27-1.12 (m, 2H), 1.08-1.01 (m, 1H). ¹³C NMR (125 MHz, CD₃OD): δ 159.3, 140.5, 133.0, 130.4, 122.7, 81.6, 57.5, 49.7, 45.4, 30.1, 28.6, 25.7, 24.2, 19.9. HRMS (ESI): m/z calcd for C₁₋₈ H₂,N₂O₂Br [M+H]⁺ 383.1329, found 383.1338. HPLC (Daicel Chiralpak IC-3, hexane/i-PrOH/2-aminoethanol 90:10:0.1, flow rate=1.0 mL/min, λ=220 nm): t_{R} 7.6 min (minor enantiomer), 11.0 min (major enantiomer).

Compound ent-5

[α]²⁵_{D} −49.2 (c 0.4, MeOH, er 0.1:>99.9). HPLC (Daicel Chiralpak IC-3, hexane/i-PrOH/2-aminoethanol 90:10:0.1, flow rate=1.0 mL/min, λ=220 nm): t_{R} 7.2 min (major enantiomer).

### Compound 5 and the Diastereomer (Dr 3:1)

¹H NMR (400 MHz, CD₃OD): δ 7.58-7.42 (m, 2H), 7.38-7.29 (m, 2H×¼), 7.23 (d, J=8.3 Hz, 2H×¾), 4.44 (d, J=10 Hz, 1H×¼), 4.29 (d, J=11.8 Hz, 1H×¾), 3.81-3.61 (m, 1H×¾), 3.19-3.00 (m, 1H×¼), 2.20-1.92 (m, 2H), 1.92-0.84 (m, 17H).

### (Synthesis of Compound 6)

K₂CO₃ (6.29 g, 45.5 mmol) and 1,5-dibromopentane (6.15 mL, 45.5 mmol) were added to a solution of Compound 5 (single diastereomer, er 99.5:0.5, 5.82 g, 15.2 mmol) in CH₃CN (100 mL) at room temperature (25° C.), and the mixture was refluxed at 60° C. (oil bath) for 18 h. The mixture was cooled to room temperature (25° C.), diluted with water, and extracted with EtOAc (50 mL×3). Organic layers were combined, dried over Na₂SO₄, filtered, and concentrated. The residue was dissolved in MeOH (20 mL), and the generated solid was collected by filtration to give Compound 6 (4.41 g, 64%, single diastereomer) as a colorless solid.

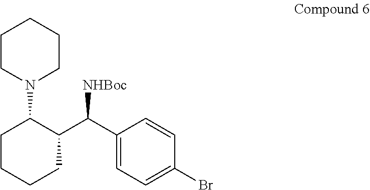 [SMILES: CC(C)(C)OC(=O)N[C@@H](c1ccc(Br)cc1)[C@@H]1CCCC[C@@H]1N1CCCCC1]

¹H NMR (500 MHz, CDCl₃): δ 9.79 (s, 1H), 7.38 (d, J=8.5 Hz, 2H), 7.18 (d, J=8.5 Hz, 2H), 4.62 (d, J=6.5 Hz, 1H), 3.03-2.70 (brm, 2H), 2.35-2.28 (m, 1H), 2.05-1.95 (m, 1H), 2.19-1.90 (m, 1H), 1.90-1.55 (m, 8H), 1.55-1.45 (m, 1H), 1.45-1.10 (m, 15H). ¹³C NMR (125 MHz, CDCl₃): δ 156.6, 143.7, 131.0, 128.6, 120.0, 78.0, 67.0, 55.3, 53.5, 49.6, 40.0, 28.3, 27.1, 26.3, 25.9, 24.3, 23.6, 20.6. HRMS (ESI): m/z calcd for C₂₃H₃₆N₂O₂Br [M+H]⁺ 451.1955, found 451.1966.

Compound 6 was crystallized from MeOH for X-ray crystal structural analysis. Colorless crystals, mp 145-150° C.

(Synthesis of Compound 1a·CF₃COOH)

CF₃COOH (15 mL) was added to a solution of Compound 6 (3.50 g, 7.76 mmol) in CH₂Cl₂ (3 mL), at room temperature (25° C.), and the mixture was stirred for 5 h at the same temperature. The mixture was concentrated, and a saturated aqueous NaHCO₃ solution was added (resulting pH~7). The mixture was extracted with EtOAc (50 mL×3). Organic layers were combined, dried over Na₂SO₄, filtered, concentrated, and purified by flash column chromatography (CH₂Cl₂/MeOH=95:5) to give Compound 1a·CF₃COOH (1:1) (2.52 g, 70%, single diastereomer, er>99.9:0.1) as a colorless solid. The ratio between Compound 1a and CF₃COOH was determined to be 1:1 by ¹³C NMR analysis using inverse gated ¹H decoupling mode (pulse program: zgig30, D1: 30 sec).

[α]²⁵_{D} −11.5 (c 1.0, MeOH, er>99.9:0.1). ¹H NMR (500 MHz, CD₃OD): δ 7.57 (d, J=8.5 Hz, 2H), 7.38 (d, J=8.5 Hz, 2H), 4.39 (d, J=11.5 Hz, 1H), 3.81-3.69 (brm, 1H), 3.50-3.37 (brm, 1H), 3.12 (dt, J=12.5 Hz, 3.5 Hz, 1H), 2.69-2.49 (m, 3H), 2.13-2.07 (m, 1H), 2.01-1.19 (m, 13H). ¹³C NMR (125 MHz, CD₃OD): δ 162.9 (q, J_{C,F}=34 Hz), 141.7, 133.2, 130.2, 122.9, 118.2 (q, J_{C,F}=290 Hz), 69.2, 55.0, 53.6, 37.4, 28.6, 26.4, 26.1, 25.7, 24.1, 23.7, 20.1. HRMS (ESI): m/z calcd for C₁₋₈ H₂₈N₂Br [M+H]⁺ 351.1430, found 351.1438. HPLC (Daicel Chiralpak IC-3, hexane/i-PrOH/2-aminoethanol 90:10:0.1, flow rate=1.0 mL/min, λ=220 nm): t_{R} 5.9 min (major enantiomer). For (±)-1a·CF₃COOH, HPLC (the same conditions): t_{R} 5.8 min and 7.1 min.

Compound 1a·CF₃COOH was crystallized from EtOAc for X-ray crystal structural analysis. Colorless crystals, mp 190-195° C.

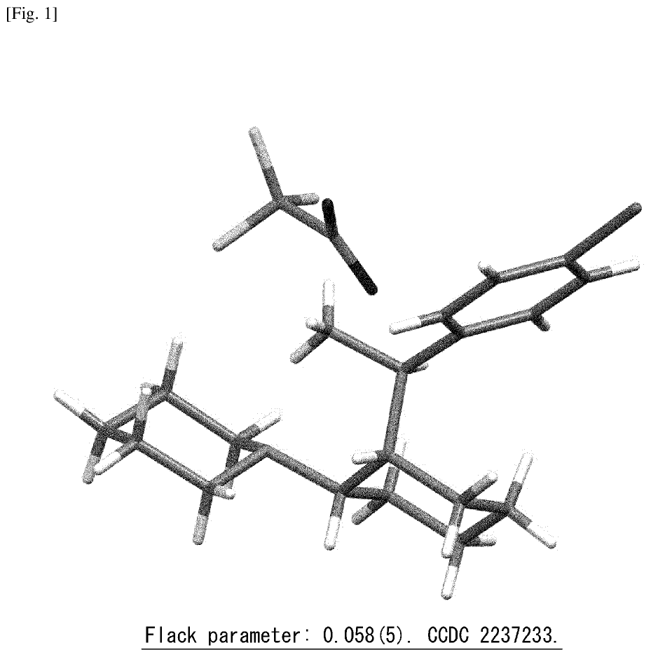

The X-ray crystal structure of Compound 1a·CF₃COOH is illustrated in FIG. 1.

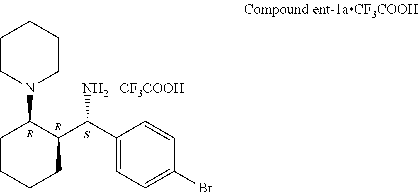

[α]²⁵_{D} +4.4 (c 1.0, MeOH, er 0.1:>99.9). HPLC (Daicel Chiralpak IC-3, hexane/i-PrOH/2-aminoethanol 90:10:0.1, flow rate=1.0 mL/min, λ=220 nm): t_{R} 8.1 mi (major enantiomer). For (±)-1a·CF₃COOH, HPLC (the same conditions): t_{R} 6.1 min and 7.8 min.

### (Formation of Compound 1a (Free Form))

A mixture of 1a·CF₃COOH (single diastereomer, 50 mg, 0.1 mmol) and a saturated aqueous NaHCO₃ solution (15 mL) (resulting pH~8) was stirred at room temperature (25° C.) for 10 min. The mixture was extracted with CH₂Cl₂ (20 mL×3). Organic layers were combined, washed with saturated aqueous NaCl solution (15 mL), dried over Na₂SO₄, filtered, and concentrated to give Compound 1a (free form) (35 mg, 90%, single diastereomer, er>99.9:0.1) as a colorless solid.

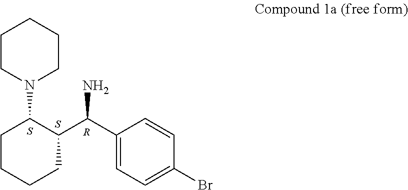

[α]²⁵_{D} +2.95 (c 0.5, MeOH, er>99.9:0.1). ¹H NMR (500 MHz, CD₃OD): δ 7.48 (d, J=8.5 Hz, 2H), 7.30 (d, J=8.5 Hz, 2H), 4.20 (d, J=10.4 Hz, 1H), 3.05-2.42 (brm, 3H), 2.59 (dt, J=10.8 Hz, 3.8 Hz, 1H), 2.38-2.28 (m, 1H), 1.93-1.85 (m, 1H), 1.85-1.78 (m, 1H), 1.78-1.71 (m, 1H), 1.71-1.55 (m, 4H), 1.55-1.41 (m, 2H), 1.41-1.05 (m, 6H). ¹³C NMR (125 MHz, CD₃OD): δ 144.4, 132.6, 130.7, 121.8, 67.7, 56.4, 53.0, 41.5, 29.4, 27.5, 26.7, 25.3, 25.0, 21.8. HPLC (Daicel Chiralpak IC-3, hexane/i-PrOH/2-aminoethanol 90:10:0.1, flow rate=1.0 mL/min, λ=220 nm): t_{R} 5.9 min (major enantiomer). For (±)-1a·CF₃COOH (1:1), HPLC (the same conditions): t_{R} 5.8 min and 7.1 min; the HPLC eluent included 2-aminoethanol and thus the CF₃COOH salt form of Compound 1a and free form of Compound 1a afforded the same retention time.

### 2. Synthesis of Compound Ent-1a

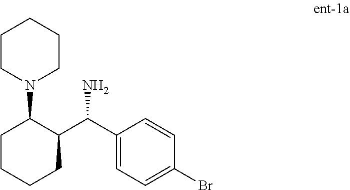 [SMILES: N[C@H](c1ccc(Br)cc1)[C@H]1CCCC[C@H]1N1CCCCC1]

Compound ent-1a was synthesized by the same procedures as those used for the synthesis of Compound 1a but using (R)-3-pyrrolidinecarboxylic acid in the formation of the enantiomer of Compound 2 instead of (S)-3-pyrrolidinecarboxylic acid in the formation of Compound 2.

### 3. Synthesis of Compound 1b

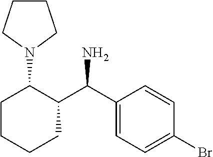 [SMILES: N[C@@H](c1ccc(Br)cc1)[C@@H]1CCCC[C@@H]1N1CCCC1]

Compound 1b was synthesized as illustrated in Scheme 5.

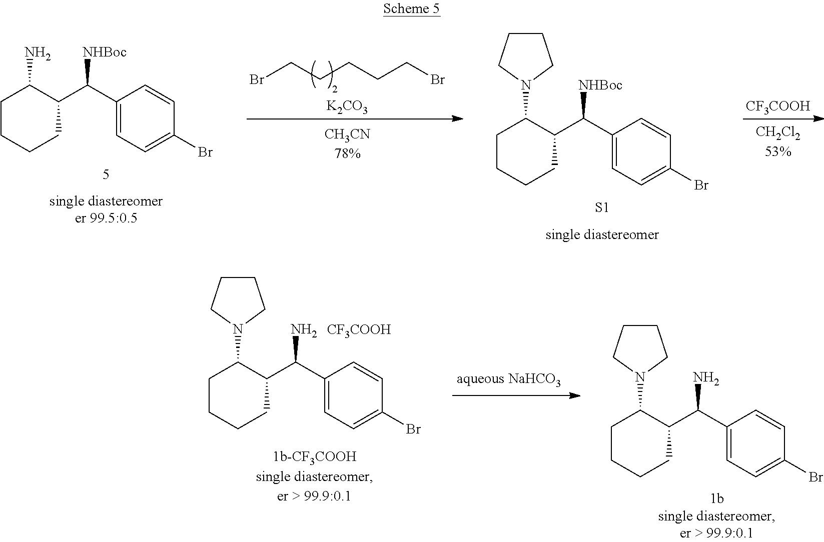

### (Synthesis of Compound S1)

Compound 5 was synthesized by the same procedures as those used for the synthesis of Compound 1a. K₂CO₃ (853 mg, 6.17 mmol) and 1,4-dibromobutane (728 μL, 6.17 mmol) were added to a solution of Compound 5 (single diastereomer, er 99.5:0.5, 800 mg, 2.05 mmol) in CH₃CN (15 mL) at room temperature (25° C.), and the mixture was refluxed at 60° C. (oil bath) for 24 h. The mixture was cooled to room temperature (25° C.), diluted with water, and extracted with EtOAc (10 mL×3). Organic layers were combined, dried over Na₂SO₄, filtered, concentrated, and purified by column chromatography (CH₂Cl₂/MeOH=97:3) to give Compound S1 (708 mg, 78%, single diastereomer) as a colorless oil.

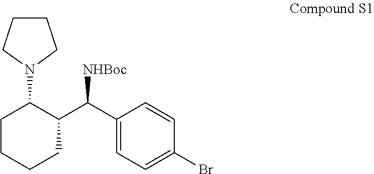 [SMILES: CC(C)(C)OC(=O)N[C@@H](c1ccc(Br)cc1)[C@@H]1CCCC[C@@H]1N1CCCC1]

¹H NMR (500 MHz, CDCl₃): δ 9.87 (s, 1H), 7.40 (d, J=8.2 Hz, 2H), 7.22 (d, J=8.2 Hz, 2H), 4.80-4.50 (brs, 1H), 3.00-2.00 (m, 5H), 2.10-2.00 (m, 1H), 1.90-1.74 (m, 5H), 1.74-1.50 (m, 2H), 1.50-1.00 (m, 14H). ¹³C NMR (125 MHz, CDCl₃): δ 157.0, 144.2, 131.1, 128.7, 120.1, 78.0, 68.3, 55.6, 51.6, 40.5, 28.3, 26.9, 25.8, 25.7, 23.2, 20.2. HRMS (ESI): m/z calcd for C₂₂H₃₄N₂O₂Br [M+H]⁺ 437.1798, found 437.1800.

(Synthesis of Compound 1b·CF₃COOH)

CF₃COOH (1.0 mL) was added to a solution of compound S1 (single diastereomer, 700 mg, 1.6 mmol) in CH₂Cl₂ (500 μL) at room temperature (25° C.), and the mixture was stirred for 1 h at the same temperature. The mixture was concentrated, and a saturated aqueous NaHCO₃ solution was added (resulting pH~7). The mixture was extracted with EtOAc (10 mL×3). Organic layers were combined, dried over Na₂SO₄, filtered, concentrated, and purified by flash column chromatography (CH₂Cl₂/MeOH=95:5) to give Compound 1b·CF₃COOH (1:1) (385 mg, 53%, single diastereomer, er>99.9:0.1) as a colorless solid. The ratio between Compound 1b and CF₃COOH was determined to be 1:1 by ¹³C NMR analysis using inverse gated ¹H decoupling mode (pulse program: zgig30, D1: 30 sec).

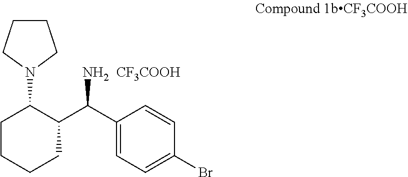 [SMILES: N[C@@H](c1ccc(Br)cc1)[C@@H]1CCCC[C@@H]1N1CCCC1 ; "CF3COOH"]

[α]^{24D} −1.3 (c 1.0, MeOH, er>99.9:0.1). ¹H NMR (500 MHz, CD₃OD): δ 7.57 (d, J=8.5 Hz, 2H), 7.37 (d, J=8.5 Hz, 2H), 4.48 (d, J=12.0 Hz, 1H), 3.50-2.85 (brm, 4H), 3.15 (dt, J=12.5 Hz, 3.5 Hz, 1H), 2.61-2.55 (m, 1H), 2.10-1.90 (m, 7H), 1.48-1.18 (m, 5H). ¹³C NMR (125 MHz, CD₃OD): δ 163.0 (q, J_{C,F}=34 Hz), 141.7, 133.2, 130.2, 123.0, 118.2 (q, J_{C,F}=291 Hz), 69.4, 55.3, 52.4, 38.8, 28.3, 26.2, 25.6, 23.7, 19.9. HRMS (ESI): m/z calcd for C₁₇H₂₆N₂Br [M+H]⁺ 337.1274, found 337.1295. HPLC (Daicel Chiralpak IC-3, hexane/i-PrOH/2-aminoethanol 90:10:0.1, flow rate=1.0 mL/min, λ=220 nm): t_{R} 5.8 min (major enantiomer). For (±)-1b·CF₃COOH (1:1), HPLC (the same conditions): t_{R} 5.8 min and 6.8 min.

### (Formation of Compound 1b (Free Form))

A mixture of Compound 1b·CF₃COOH (single diastereomer, 20 mg, 0.044 mmol) and a saturated aqueous NaHCO₃ solution (10 mL) (resulting pH~8) was stirred at room temperature (25° C.) for 10 min. The mixture was extracted with CH₂Cl₂ (20 mL×3). Organic layers were combined, washed with saturated aqueous NaCl solution (10 mL), dried over Na₂SO₄, filtered, and concentrated to give Compound 1b (free form) (12 mg, 80%, single diastereomer) as a pale yellow solid.

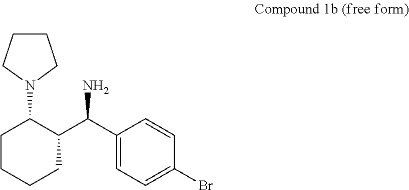 [SMILES: N[C@@H](c1ccc(Br)cc1)[C@@H]1CCCC[C@@H]1N1CCCC1]

[α]²⁵, +1092.8 (c 1.0, MeOH, er (based on compound 1b·CF₃COOH)>99.9:0.1). ¹H NMR (500 MHz, CD₃OD): δ 7.49 (d, J=8.5 Hz, 2H), 7.32 (d, J=8.5 Hz, 2H), 4.35 (d, J=11.0 Hz, 1H), 3.06-2.76 (m, 2H), 2.74-2.57 (m, 2H), 2.53 (dt, J=12.0 Hz, 3.5 Hz, 1H), 2.39-2.31 (m, 1H), 1.99-1.72 (m, 7H), 1.49-1.26 (m, 2H), 1.26-1.07 (m, 3H). ¹³C NMR (125 MHz, CD₃OD): δ 144.5, 132.7, 130.6, 121.9, 70.1, 56.1, 53.4, 42.1, 29.3, 27.1, 26.7, 24.0, 21.0.

### 4. Synthesis of Compound 1c

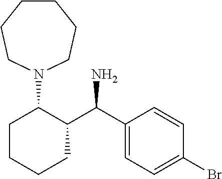 [SMILES: N[C@@H](c1ccc(Br)cc1)[C@@H]1CCCC[C@@H]1N1CCCCCC1]

Compound 1c was synthesized as illustrated in Scheme 6.

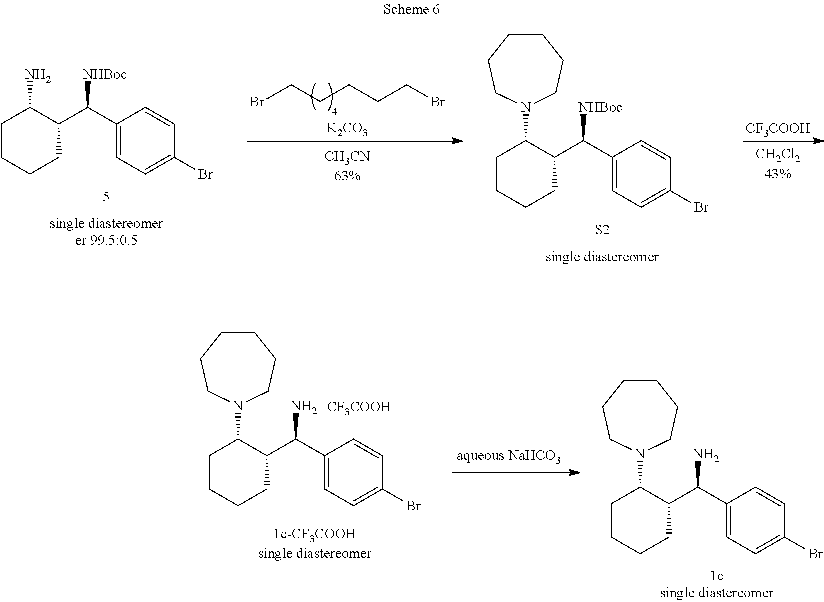

### (Synthesis of Compound S2)

Compound 5 was synthesized by the same procedures as those used for the synthesis of compound 1a. K₂CO₃ (212 mg, 1.54 mmol) and 1,6-dibromohexane (220 μL, 1.54 mmol) were added to a solution of Compound 5 (single diastereomer, er 99.5:0.5, 200 mg, 0.514 mmol) in CH₃CN (5 mL) at room temperature (25° C.), and the mixture was refluxed at 60° C. (oil bath) for 24 h. The mixture was cooled to room temperature (25° C.), diluted with water, and extracted with EtOAc (10 mL×3). Organic layers were combined, dried over Na₂SO₄, filtered, concentrated, and purified by column chromatography (CH₂Cl₂/MeOH=97:3) to give Compound S2 (152 mg, 63%, single diastereomer) as a colorless oil.

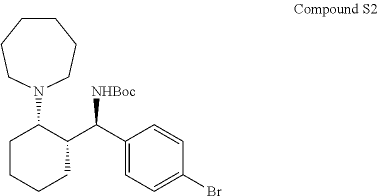 [SMILES: CC(C)(C)OC(=O)N[C@@H](c1ccc(Br)cc1)[C@@H]1CCCC[C@@H]1N1CCCCCC1]

¹H NMR (500 MHz, CD₃OD): δ 7.46 (d, J=8.5 Hz, 2H), 7.18 (d, J=8.5 Hz, 2H), 4.50 (d, J=7.5 Hz, 1H), 3.47 (t, J=6.5 Hz, 2H), 2.78-2.68 (m, 2H), 2.36-2.28 (m, 1H), 1.95-1.15 (m, 26H). ¹³C NMR (125 MHz, CD₃OD): δ 157.8, 143.6, 132.3, 129.8, 121.4, 80.0, 58.7, 54.4, 47.4, 34.4, 33.9, 30.9, 29.5, 29.1, 28.8, 27.7, 27.0, 26.2, 20.6. HRMS (ESI): m/z calcd for C₂₄H₃₈N₂O₂Br [M+H]⁺ 465.2111, found 465.2105.

(Synthesis of Compound 1c·CF₃COOH)

CF₃COOH (2.4 mL) was added to a solution of Compound S2 (single diastereomer, 150 mg, 0.32 mmol) in CH₂Cl₂ (1.2 mL) at room temperature (25° C.), and the mixture was stirred for 1 h at the same temperature. The mixture was concentrated, and a saturated aqueous NaHCO₃ solution was added (resulting pH~7). The mixture was extracted with EtOAc. Organic layers were combined, dried over Na₂SO₄, filtered, concentrated, and purified by flash column chromatography (CH₂Cl₂/MeOH=95:5) to give Compound 1c·CF₃COOH (65 mg, 43%, single diastereomer) as a colorless solid.

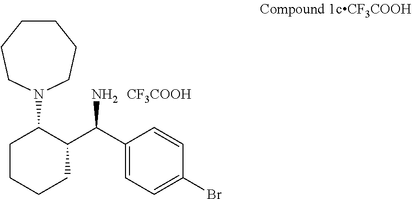 [SMILES: N[C@@H](c1ccc(Br)cc1)[C@@H]1CCCC[C@@H]1N1CCCCCC1 ; "CF3COOH"]

¹H NMR (500 MHz, CD₃OD): δ 7.55 (d, J=8.5 Hz, 2H), 7.33 (d, J=8.5 Hz, 2H), 4.33 (d, J=10.0 Hz, 1H), 3.47 (t, J=6.5 Hz, 2H), 3.25 (dt, J=11.0 Hz, 3.5 Hz, 1H), 3.12 (dt, J=11.0 Hz, 8.0 Hz, 1H), 2.97-2.90 (m, 1H), 2.40-2.34 (m, 1H), 2.04-1.82 (m, 5H), 1.74-1.64 (m, 2H), 1.59-1.23 (m, 9H). ¹³C NMR (125 MHz, CD₃OD): δ 162.9 (q, J_{C,F}=33.7 Hz), 143.2, 133.1, 129.8, 122.5, 118.2 (q, J_{C,F}=291 Hz), 61.5, 55.9, 46.9, 39.2, 34.2, 33.6, 28.8, 28.1, 27.9, 26.9, 26.8, 24.9, 21.2. HRMS (ESI): m/z calcd for C₁₉H₃₀N₂Br [M+H]⁺ 365.1587, found 365.1581.

### (Formation of Compound 1c (Free Form))

A mixture of 1c·CF₃COOH (single diastereomer, 15 mg, 0.031 mmol) and a saturated aqueous NaHCO₃ solution (5 mL) (resulting pH~8) was stirred at room temperature (25° C.) for 10 min. The mixture was extracted with CH₂Cl₂ (10 mL×3). Organic layers were combined, washed with saturated aqueous NaCl solution (5 mL), dried over Na₂SO₄, filtered, and concentrated to give Compound 1c (free form) (10 mg, 88%, single diastereomer) as a colorless solid.

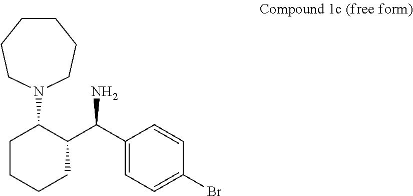 [SMILES: N[C@@H](c1ccc(Br)cc1)[C@@H]1CCCC[C@@H]1N1CCCCCC1]

[α]²⁵_{D} +2.52 (c 0.5, MeOH, er (based on compound 5) 99.5:0.5). ¹H NMR (500 MHz, CD₃OD): δ 7.49 (d, J=8.4 Hz, 2H), 7.26 (d, J=8.5 Hz, 2H), 3.97 (d, J=9.7 Hz, 1H), 3.50-3.43 (m, 2H), 3.09-3.03 (m, 1H), 2.86-2.77 (m, 1H), 2.68-2.58 (m, 1H), 2.06-1.94 (m, 1H), 1.93-1.77 (m, 3H), 1.69-1.05 (m, 13H). ¹³C NMR (125 MHz, CD₃OD): β 144.9, 132.6, 130.2, 121.8, 57.8, 56.5, 47.7, 45.9, 34.3, 33.8, 30.3, 29.0, 28.4, 27.5, 26.7, 25.0, 22.3.

### 5. Synthesis of Compound 1d

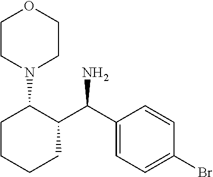 [SMILES: N[C@@H](c1ccc(Br)cc1)[C@@H]1CCCC[C@@H]1N1CCOCC1]

Compound 1d was synthesized as illustrated in Scheme 7.

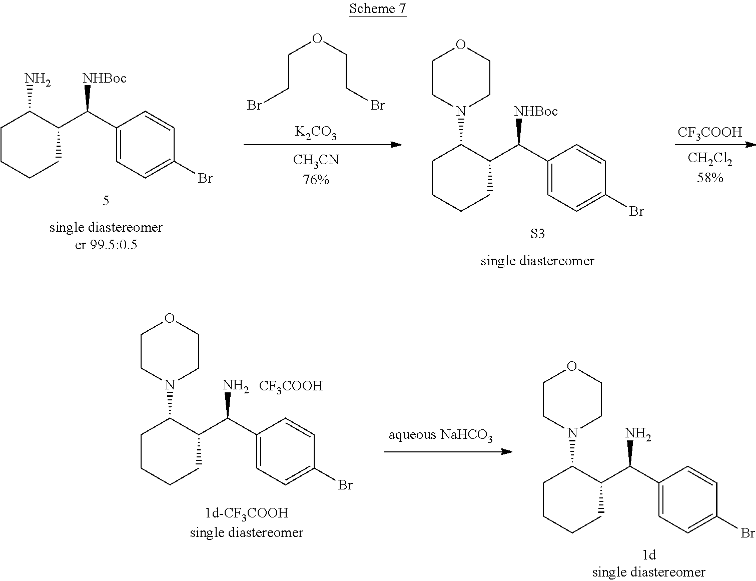

### (Synthesis of Compound S3)

Compound 5 was synthesized by the same procedures as those used for the synthesis of compound 1a. K₂CO₃ (960 mg, 6.94 mmol) and bis(2-bromoethyl)ether (865 μL, 6.94 mmol) were added to a solution of compound 5 (single diastereomer, er 99.5:0.5, 900 mg, 2.31 mmol) in CH₃CN (20 mL) at room temperature (25° C.), and the mixture was refluxed at 60° C. (oil bath) for 24 h. The mixture was cooled to room temperature (25° C.), diluted with water, and extracted with EtOAc (10 mL×3). Organic layers were combined, dried over Na₂SO₄, filtered, concentrated, and purified by column chromatography (CH₂Cl₂/MeOH=97:3) to give Compound S3 (810 mg, 76%, single diastereomer) as a colorless solid.

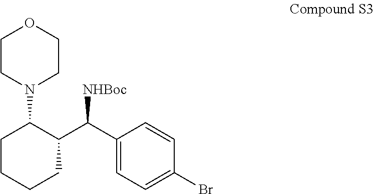 [SMILES: CC(C)(C)OC(=O)N[C@@H](c1ccc(Br)cc1)[C@@H]1CCCC[C@@H]1N1CCOCC1]

¹H NMR (500 MHz, CD₃OD): δ 7.45 (d, J=8.5 Hz, 2H), 7.23 (d, J=8.5 Hz, 2H), 4.65 (d, J=10.0 Hz, 1H), 3.85-3.72 (m, 4H), 2.80-2.43 (m, 5H), 2.15-2.07 (m, 1H), 1.93-1.78 (m, 2H), 1.73-1.61 (m, 1H) 1.61-1.50 (m, 1H), 1.50-1.19 (m, 13H). ¹³C NMR (125 MHz, CD₃OD): δ 158.1, 144.8, 132.3, 130.2, 121.3, 79.8, 68.4, 67.7, 56.6, 52.4, 41.3, 28.7, 28.0, 26.6, 24.5, 22.1. HRMS (ESI): m/z calcd for C₂₂H₃₄N₂O₃Br [M+H]⁺ 453.1747, found 453.1748.

(Synthesis of Compound 1d·CF₃COOH)

CF₃COOH (2.0 mL) was added to a solution of Compound S3 (single diastereomer, 400 mg, 0.88 mmol) in CH₂Cl₂ (2.0 mL) at room temperature (25° C.), and the mixture was stirred for 1 h at the same temperature. The mixture was concentrated, and a saturated aqueous NaHCO₃ solution was added (resulting pH~7). The mixture was extracted with EtOAc (10 mL×3). Organic layers were combined, dried over Na₂SO₄, filtered, concentrated, and purified by flash column chromatography (CH₂Cl₂/MeOH=95:5) to give 1d·CF₃COOH (1:1) (240 mg, 58%, single diastereomer) as a colorless solid.

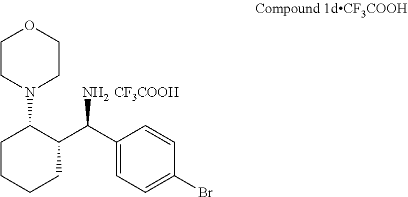 [SMILES: N[C@@H](c1ccc(Br)cc1)[C@@H]1CCCC[C@@H]1N1CCOCC1 ; "CF3COOH"]

¹H NMR (500 MHz, CDCl₃): δ 9.36 (brs, 2H), 7.46 (d, J=8.0 Hz, 2H), 7.31 (d, J=8.0 Hz, 2H), 4.41 (d, J=11.5 Hz, 1H), 3.92-3.80 (m, 2H), 3.45-3.37 (m, 1H), 3.37-3.23 (m, 2H), 2.88-2.81 (m, 1H), 2.75-2.68 (m, 1H), 2.55 (dt, J=12.5 Hz, 3.1 Hz, 1H), 2.29-2.21 (m, 1H), 2.21-2.12 (m, 1H), 1.98-1.86 (m, 2H), 1.53 (qd, J=12.5 Hz, 3.1 Hz, 1H), 1.33-1.00 (m, 5H). ¹³C NMR (125 MHz, CDCl₃): δ 161.2 (q, J_{C,F}=35 Hz), 134.4, 132.2, 129.6, 123.3, 116.4 (q, J_{C,F}=291 Hz), 67.0, 66.8, 66.4, 54.4, 51.9, 49.2, 34.9, 26.9, 25.2, 23.1, 19.1. HRMS (ESI): m/z calcd for C₁₇H₂₆N₂OBr [M+H]⁺ 353.1223, found 353.1232.

### (Formation of Compound 1d (Free Form))

A mixture of 1d·CF₃COOH (1:1) (single diastereomer, 40 mg, 0.086 mmol) and a saturated aqueous NaHCO₃ solution (resulting pH~8) was stirred at room temperature (25° C.) for 10 min. The mixture was extracted with CH₂Cl₂. Organic layers were combined, washed with saturated aqueous NaCl solution, dried over Na₂SO₄, filtered, and concentrated to give Compound 1d (free form) (25 mg, 84%, single diastereomer) as a colorless solid.

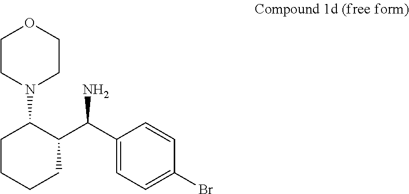 [SMILES: N[C@@H](c1ccc(Br)cc1)[C@@H]1CCCC[C@@H]1N1CCOCC1]

[α]²⁵_{D}+423.4 (c 1.0, CH₂Cl₂, er (based compound 5) 99.5:0.5). ¹H NMR (500 MHz, CDCl₃): δ 7.43 (d, J=8.5 Hz, 2H), 7.24 (d, J=8.5 Hz, 2H), 4.27 (d, J=10.0 Hz, 1H), 3.82-3.63 (m, 4H), 3.09-2.33 (m, 6H), 2.41 (dt, J=11.0 Hz, 3.6 Hz, 1H), 2.31-2.20 (m, 1H), 1.91-1.73 (m, 2H), 1.70-1.55 (m, 1H), 1.42-1.05 (m, 5H). ¹³C NMR (125 MHz, CDCl₃): δ 144.3, 131.3, 129.3, 120.5, 67.3, 66.8, 55.3, 51.7, 40.4, 28.4, 25.5, 23.7, 20.9.

### 6. Synthesis of Compound 1e

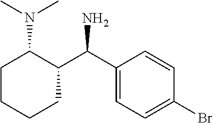 [SMILES: CN(C)[C@H]1CCCC[C@H]1[C@@H](N)c1ccc(Br)cc1]

Compound 1e was synthesized as illustrated in Scheme 8.

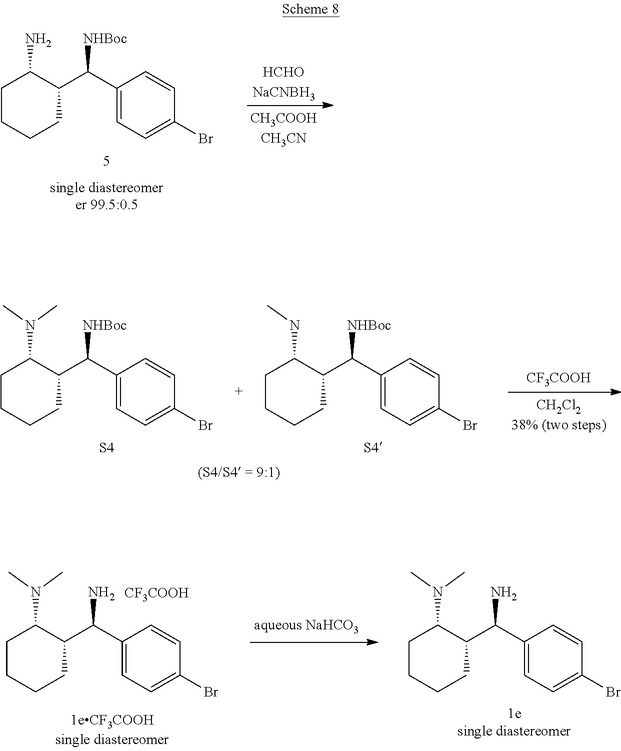

### (Synthesis of Compound S4)

Compound 5 was synthesized by the same procedures as those used for the synthesis of compound 1a. HCHO (37% (w/w) in H₂O, 1.62 mL, 20 mmol), NaCNBH₃ (377 mg, 6.0 mmol), and AcOH (343 μL, 6.0 mmol) were added to a solution of Compound 5 (single diastereomer, er 99.5:0.5, 778 mg, 2.0 mmol) in CH₃CN (8 mL), at room temperature (25° C.), and the mixture was stirred for 21 h. The mixture was diluted with water and extracted with EtOAc (20 mL×3). Organic layers were combined, dried over Na₂SO₄, filtered, concentrated, and purified by flash column chromatography (CH₂Cl₂/MeOH=97:3) to give a mixture of Compound S4 and S4′ (S4/S4′=9:1, 800 mg) as a colorless oil.

### Compounds S4 and S4′ (9:1)

¹H NMR (500 MHz, CD₃OD): δ 7.46 (d, J=8.0 Hz, 2H), 7.23 (d, J=8.0 Hz, 2H× 9/10), 7.19 (d, J=8.0 Hz, 2H× 1/10), 4.61 (d, J=10.0 Hz, 1H), 2.39 (s, 6H× 9/10), 2.23 (s, 6H, 1/10), 2.22-2.10 (m, 1H), 1.98-1.80 (m, 2H), 1.78-1.48 (m, 3H), 1.48-0.97 (m, 13H). ¹³C NMR (125 MHz, CD₃OD): δ 158.4, 132.5, 132.2, 130.2, 121.3, 80.0, 56.8, 43.7, 41.4, 30.7, 28.8, 28.7, 28.6, 28.0, 26.6, 25.5, 21.7. HRMS (ESI): m/z calcd for C₂₀H₃₂N₂O₂Br [M+H]⁺ 411.1642, found 411.1651 for compound S4.

(Synthesis of Compound 1e·CF₃COOH)

CF₃COOH (2.0 mL) was added to a solution of Compound S4 and S4′ (S4/S4′=9:1, 800 mg) in CH₂Cl₂ (1.0 mL) at room temperature (25° C.), and the mixture was stirred for 1 h at the same temperature. The mixture was concentrated, and a saturated aqueous NaHCO₃ solution was added (resulting pH~7). The mixture was extracted with EtOAc. Organic layers were combined, dried over Na₂SO₄, filtered, concentrated, and purified by flash column chromatography (CH₂Cl₂/MeOH=95:5) to give compound 1e·CF₃COOH (1:1) (280 mg, 38% for two steps from 5, single diastereomer) as a colorless oil. The ratio between Compound 1e and CF₃COOH was determined to be 1:1 by ¹³C NMR analysis using inverse gated ¹H decoupling mode (pulse program: zgig30, D1: 30 sec).

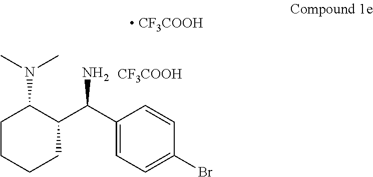 [SMILES: CN(C)[C@H]1CCCC[C@H]1[C@@H](N)c1ccc(Br)cc1 ; "CF3COOH" ; "· CF3COOH"]

¹H NMR (500 MHz, CD₃OD): δ 7.58 (d, J=8.5 Hz, 2H), 7.39 (d, J=8.5 Hz, 2H), 4.47 (d, J=11.5 Hz, 1H), 2.96 (dt, J=13.0 Hz, 3.5 Hz, 1H), 2.72-2.65 (m, 1H), 2.65 (s, 6H), 2.13-2.07 (m, 1H), 2.02-1.93 (m, 1H), 1.86 (qd, J=12.5 Hz, 3.8 Hz, 1H), 1.44-1.20 (m, 5H). ¹³C NMR (125 MHz, CD₃OD): δ 162.9 (q, J_{C,F}=34 Hz), 140.8, 133.3, 130.4, 123.2, 118.2 (q, J_{C,F}=291 Hz), 70.0, 55.4, 41.7, 37.3, 28.4, 26.4, 24.8, 19.9. HRMS (ESI): m/z calcd for C₁₅H₂₄N₂Br [M+H]⁺ 311.1117, found 311.1126.

### (Formation of Compound 1e (Free Form))

A mixture of 1e·CF₃COOH (1:1) (single diastereomer, 20 mg, 0.047 mmol) and a saturated aqueous NaHCO₃ solution (15 mL) (resulting pH~8) was stirred at room temperature (25° C.) for 10 min. The mixture was extracted with CH₂Cl₂. Organic layers were combined, washed with saturated aqueous NaCl solution, dried over Na₂SO₄, filtered, and concentrated to give Compound 1e (free form) (11 mg, 70%, single diastereomer) as a colorless solid.

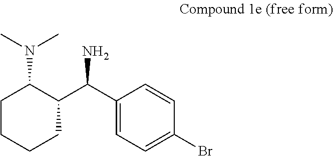 [SMILES: CN(C)[C@H]1CCCC[C@H]1[C@@H](N)c1ccc(Br)cc1]

[α]²⁵_{D} −11.5 (c 0.5, MeOH, er (based on compound 5) 99.5:0.5). ¹H NMR (500 MHz, CD₃OD): δ 7.49 (d, J=8.4 Hz, 2H), 7.31 (d, J=8.4 Hz, 2H), 4.27 (d, J=10.2 Hz, 1H), 2.38 (s, 6H), 2.42-2.33 (m, 2H), 1.99-1.89 (m, 1H), 1.89-1.79 (m, 1H), 1.79-1.65 (m, 1H), 1.43-1.24 (m, 3H), 1.24-1.08 (m, 3H). ¹³C NMR (125 MHz, CD₃ OD): δ 144.6, 132.6, 130.7, 121.8, 69.7, 56.4, 44.1, 41.3, 29.3, 26.8, 25.6, 21.3.

### 7. Synthesis of Compound 1f

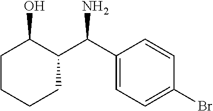 [SMILES: N[C@@H](c1ccc(Br)cc1)[C@@H]1CCCC[C@H]1O]

Compound 1f was synthesized as illustrated in Scheme 9.

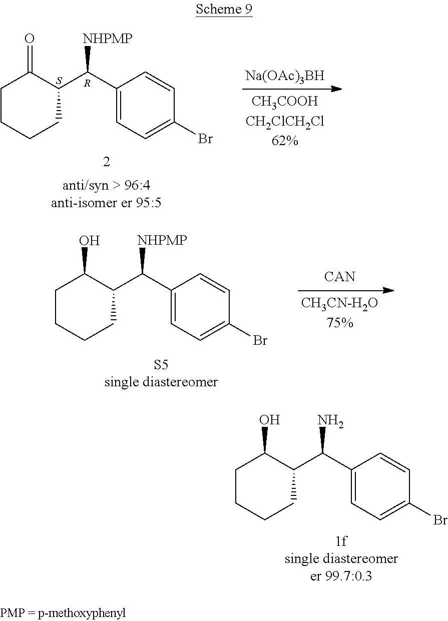

### (Synthesis of Compound S5)

Compound 2 was synthesized by the same procedures as those used for the synthesis of Compound 1a. Na(OAc)₃BH (21.8 g, 103 mmol) and CH₃COOH (5.9 mL, 103 mmol) were added to a solution of Compound 2 (anti/syn >96:4, er 95:5, 8.00 g, 20.6 mmol) in 1,2-dichloroethane (250 mL) at 0° C., and the mixture was stirred at the same temperature for 24 h (progress was monitored by TLC and ¹H NMR). Further, Na(OAc)₃BH (7.27 g, 34.3 mmol) and CH₃COOH (5.89 mL, 103 mmol) were added, and the mixture was stirred at the same temperature for another 24 h (progress was monitored by TLC and ¹H NMR). Additionally, Na(OAc)₃BH (7.27 g, 34.3 mmol) and AcOH (5.89 mL, 103 mmol) were added, and the mixture was stirred for another 24 h. The mixture was quenched by the addition of a saturated aqueous NaHCO₃ solution and extracted with CH₂Cl₂ (100 mL×3). The organic layers were combined, dried over Na₂SO₄, filtered, concentrated, and purified by flash column chromatography (hexane/EtOAc=60:40) to give Compound S5 (4.98 g, 62%, single diastereomer) as a colorless oil.

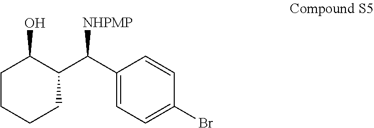

¹H NMR (500 MHz, CDCl₃): δ 7.39 (d, J=8.5 Hz, 2H), 7.08 (d, J=8.5 Hz, 2H), 6.67 (d, J=8.5 Hz, 2H), 6.59 (d, J=8.5 Hz, 2H), 4.23 (d, J=8.5 Hz, 1H), 3.69 (s, 3H), 3.61 (td, J=9.5 Hz, 4.5 Hz, 1H), 2.03-2.00 (m, 1H), 1.75-1.67 (m, 2H), 1.60-1.56 (m, 1H), 1.38-1.16 (m, 3H), 1.13-1.04 (m, 1H), 0.73 (qd, J=12.8 Hz, 3.6 Hz, 1H). ¹³C NMR (125 MHz, CDCl₃): δ 153.9, 140.0, 139.1, 131.6, 128.9, 121.0, 118.3, 118.2, 114.5, 75.5, 66.3, 55.5, 49.0, 35.4, 28.1, 25.4, 24.4. HRMS (ESI): m/z z calcd for C₂₀H₂₅NO₂Br [M+H]⁺ 390.1063, found 390.1040.

### (Synthesis of Compound 1f)

A solution of ammonium cerium nitrate (CAN) (3.64 g, 6.64 mmol) in H₂O (22.4 mL) to a solution of Compound S5 (589 mg, 1.51 mmol) in CH₃CN (38 mL) was added at 0° C., and the mixture was stirred at the same temperature for 2 h. The mixture was diluted with water (100 mL) and extracted with EtOAc (30 mL×3). Organic layers were combined, dried over Na₂SO₄, filtered, concentrated, and purified by flash column chromatography (CH₂Cl₂/MeOH=90:10) to give Compound 1f (323 mg, 75%, single diastereomer, er 96:4) as a colorless solid. The compound was crystallized from EtOAc/MeOH (99:1 (v/v)) to give Compound 1f (180 mg, single diastereomer, er 99.7:0.3) as colorless crystals. The relative stereochemistry of Compound 1f was determined by NMR analyses based on coupling constants (J values), COSY, NOESY, and HSQC.

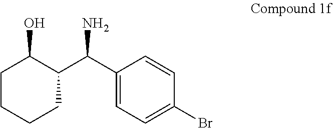 [SMILES: N[C@@H](c1ccc(Br)cc1)[C@@H]1CCCC[C@H]1O]

[α]²⁵_{D} −42.8 (c 0.5, MeOH, er 99.7:0.3). ¹H NMR (500 MHz, CD₃OD): δ 7.62 (d, J=8.0 Hz, 2H), 7.34 (d, J=8.0 Hz, 2H), 4.32 (d, J=9.0 Hz, 1H), 3.53 (td, J=10.0 Hz, 4.0 Hz, 1H), 2.01-1.96 (m, 1H), 1.86-1.79 (m, 1H), 1.75-1.69 (m, 1H), 1.62-1.56 (m, 1H), 1.43-1.08 (m, 4H), 0.82 (dq, J=12.5 Hz, 3.7 Hz, 1H). ¹³C NMR (125 MHz, CD₃ OD): δ 136.4, 133.3, 130.9, 124.1, 75.9, 60.9, 47.7, 36.9, 28.6, 26.1, 25.4. HRMS (ESI): m/z calcd for C₁₃H₁₉NOBr [M+H]⁺ 284.0645, found 284.0629. HPLC (Daicel Chiralpak AS-3, hexane/i-PrOH=90:10, flow rate=1.0 mL/min, λ=220 nm): t_{R} 10.5 min (major enantiomer), 12.0 min (minor enantiomer).

### 8. Synthesis of Compound 1h

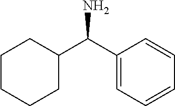 [SMILES: N[C@@H](c1ccccc1)C1CCCCC1]

Compound 1h was synthesized as illustrated in Scheme 10.

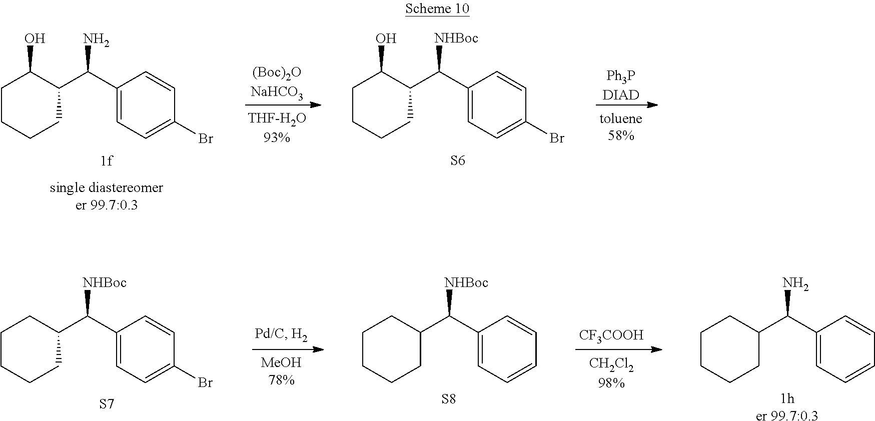

### (Synthesis of Compound S6)

Compound 1f was synthesized by the same procedures as those used for the synthesis of Compound 1f. NaHCO₃ (444 mg, 5.28 mmol) and di-tert-butyl decarbonate (485 μL, 2.11 mmol) were added to a solution of compound 1f (single diastereomer, er 99.7:0.3, 500 mg, 1.76 mmol) in THF/H₂O (1:1 (v/v), 10 mL) at 0° C., and the mixture was stirred at room temperature (25° C.) for 5.5 h. The mixture was concentrated to remove the organic solvent and extracted with EtOAc (50 mL×3). Organic layers were combined, dried over Na₂SO₄, concentrated, and purified by flash column chromatography (hexane/EtOAc=70:30) to give Compound S6 (630 mg, 93%, single diastereomer) as a colorless oil.

### Compound S6

¹H NMR (500 MHz, CDCl₃): δ 7.42 (d, J=8.2 Hz, 2H), 7.15 (d, J=8.2 Hz, 2H), 5.64 (brs, 1H), 4.82 (apps, 1H), 3.28 (td, J=10.0 Hz, 4.0 Hz, 1H), 1.99-1.92 (m, 1H), 1.92-1.57 (m, 3H), 1.57-1.50 (m, 1H), 1.47-1.21 (m, 11H), 1.19-1.04 (m, 2H), 0.86-0.74 (m, 1H). ¹³C NMR (125 MHz, CDCl₃): δ 155.4, 140.4, 131.2, 129.1, 120.7, 79.6, 72.3, 56.8, 49.3, 36.2, 28.3, 26.6, 25.0, 24.4. HRMS (ESI): m/z calcd for C₁₋₈ H₂₇NO₃Br [M+H]⁺ 384.1169, found 384.1173.

### (Synthesis of Compound S7)

Ph₃P (273 mg, 1.04 mmol) and diisopropyl azodicarboxylate (DIAD) (204 μL, 1.04 mmol) were added to a solution of compound S6 (single diastereomer, 100 mg, 0.26 mmol) in toluene (10 mL), at 0° C. and the mixture was stirred at room temperature (25° C.) for 20 h. The mixture was diluted with water and extracted with EtOAc (10 mL×3). Organic layers were combined, dried over Na₂SO₄, filtered, concentrated, and purified by flash column chromatography (hexane/EtOAc=95:5) to give Compound S7 (55 mg, 58%, single diastereomer) as a colorless oil.

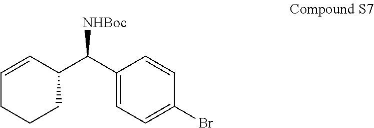 [SMILES: CC(C)(C)OC(=O)N[C@@H](c1ccc(Br)cc1)[C@H]1C=CCCC1]

¹H NMR (500 MHz, CDCl₃): δ 7.43 (d, J=8.0 Hz, 2H), 7.11 (d, J=8.0 Hz, 2H), 5.89-5.84 (m, 1H), 5.50-5.41 (m, 1H), 4.90 (d, J=6.5 Hz, 1H), 4.57 (t, J=6.5 Hz, 1H), 2.52-2.41 (m, 1H), 2.02-1.94 (m, 2H), 1.79-1.66 (m, 2H), 1.55-1.20 (m, 11H). ¹³C NMR (125 MHz, CDCl₃): δ 155.4, 141.2, 131.6, 131.3, 128.0, 125.0, 120.5, 79.5, 57.7, 41.3, 28.3, 27.1, 25.0, 21.5. HRMS (ESI): m/z calcd for C₁₈H₂₅NO₂Br [M+H]⁺ 366.1063, found 366.1070.

### (Synthesis of Compound S8)

Pd/C (10%, 30 mg) was added to a solution of Compound S7 (single diastereomer, 127 mg, 0.35 mmol) in MeOH (1.5 mL) at room temperature (25° C.), and the mixture was stirred under H₂ (balloon) for 1 h. The reaction mixture was filtered through a pad of Celite and washed with MeOH and EtOAc. The filtrate was concentrated and purified by flash column chromatography (CH₆Cl₂) to give Compound S8 (77 mg, 78%, single diastereomer) as a colorless oil.

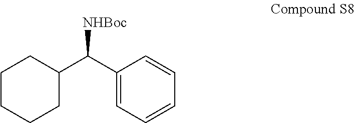 [SMILES: CC(C)(C)OC(=O)N[C@@H](c1ccccc1)C1CCCCC1]

¹H NMR (500 MHz, CDCl₃): δ 7.30 (t, J=7.4 Hz, 2H), 7.24-7.13 (m, 3H), 4.88 (s, 1H), 4.41 (apps, 1H), 1.91-1.79 (m, 1H), 1.79-1.71 (m, 1H), 1.71-1.50 (m, 3H), 1.49-1.23 (m, 10H), 1.23-1.05 (m, 3H), 1.05-0.83 (m, 2H). ¹³C NMR (125 MHz, CDCl₃): δ 155.4, 142.0, 128.3, 126.8, 79.2, 59.9, 43.6, 30.1, 29.1, 28.4, 26.2, 26.13, 26.10. HRMS (ESI): m/z calcd for C₁₋₈ H₂₈NO₂ [M+H]⁺ 290.2115, found 290.2104.

### (Synthesis of Compound 1h)

CF₃COOH (1.0 mL) was added to a solution of Compound S8 (single diastereomer, 70 mg, 0.24 mmol) in CH₂Cl₂ (2.0 mL) at room temperature (25° C.), and the mixture was stirred for 2 h at the same temperature. The mixture was concentrated, and a saturated aqueous NaHCO₃ solution was added. The mixture was extracted with EtOAc. Organic layers were combined, dried over Na₂SO₄, filtered, concentrated, and purified by flash column chromatography (CH₂Cl₂/MeOH=95:5) to give Compound 1h (44 mg, 98% single diastereomer, er 99.7:0.3) as a colorless oil.

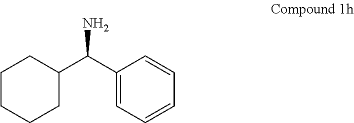 [SMILES: N[C@@H](c1ccccc1)C1CCCCC1]

[α]²⁵_{D} +5.6 (c 1.0, MeOH, er 99.7:0.3). ¹H NMR (500 MHz, CD₃OD): δ 7.32-7.20 (m, 5H), 3.50 (d, J=8.0 Hz, 1H), 2.03-1.96 (m, 1H), 1.83-1.76 (m, 1H), 1.69-1.58 (m, 2H), 1.58-1.49 (m, 1H), 1.38-1.22 (m, 2H), 1.22-1.08 (m, 2H), 1.01 (qd, J=12.4 Hz, 3.5 Hz, 1H), 0.90-0.79 (m, 1H). ¹³C NMR (125 MHz, CD₃OD): δ 145.7, 129.2, 128.3, 128.0, 62.8, 46.2, 31.1, 30.8, 27.5, 27.30, 27.26. HRMS (ESI): m/z calcd for C₁₃H₂₀N [M+H]⁺ 190.1590, found 190.1582. HPLC (Daicel Chiralpak IC-3, hexane/i-PrOH=80:20, flow rate=1.0 mL/min, λ=220 nm): t_{R} 7.0 min (major enantiomer), 8.4 min (minor enantiomer).

### 9. Synthesis of Compounds 1g and 1i

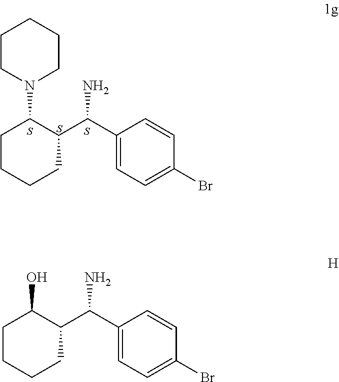

Compounds 1g and 1i were synthesized as illustrated in Scheme 11.

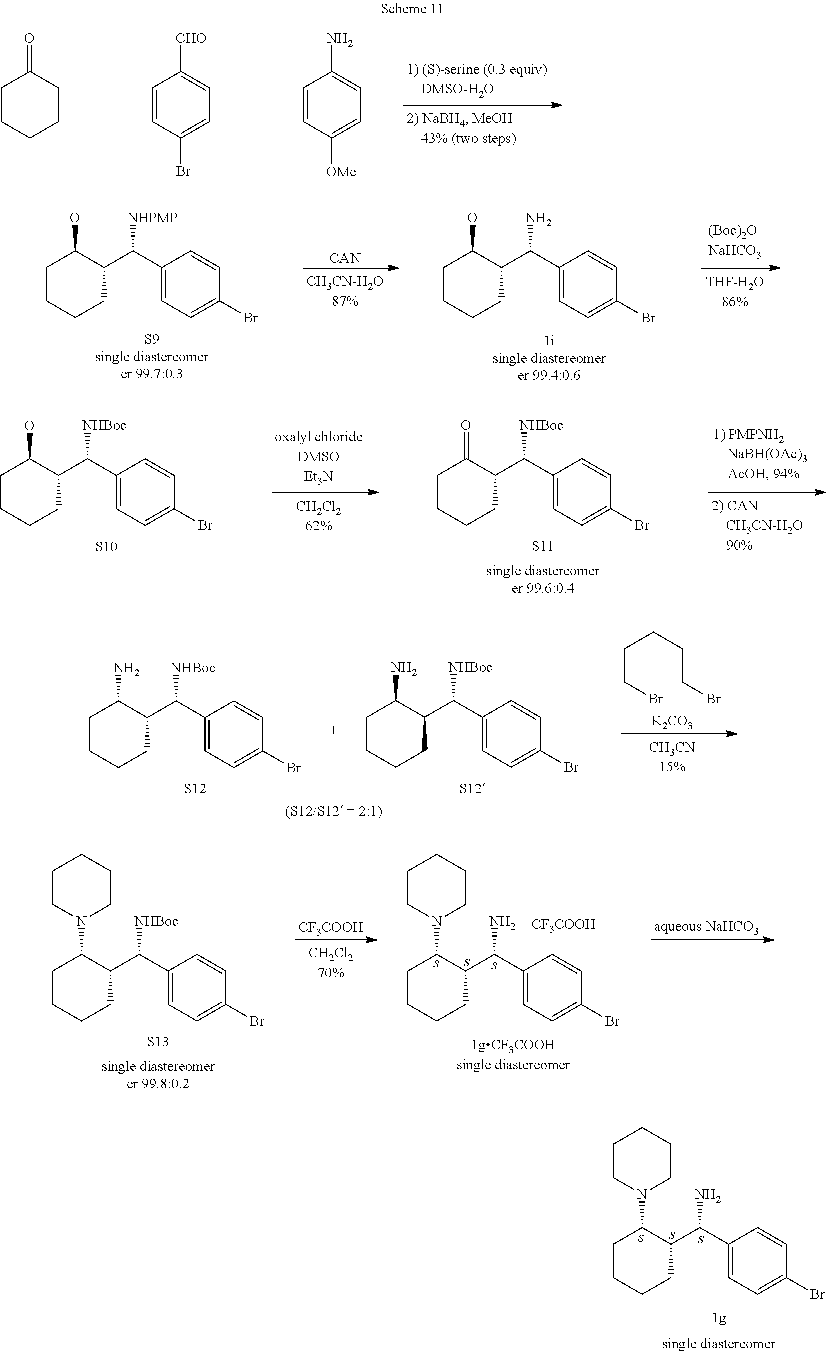

### (Synthesis of Compound S9)

p-Anisidine (739 mg, 6.00 mmol), cyclohexanone (6.21 mL, 60 mmol), and (S)-serine (189 mg, 1.80 mmol) were sequentially added to a mixture of H₂O (238 μL, 13.2 mmol) and DMSO (24 mL), 4-bromobenzaldehyde (1.22 g, 6.60 mmol), and the mixture was stirred for 72 h. The mixture was diluted with water and extracted with EtOAc (30 mL×3). Organic layers were combined, dried over Na₂SO₄, filtered, and concentrated to give the Mannich reaction product, which was used for the next step without purification.

The residue was dissolved in MeOH (15 mL), and NaBH₄ (294 mg, 7.8 mmol) was added at 0° C. The mixture was stirred for 30 min at the same temperature. The reaction mixture was quenched with saturated aqueous NH₄Cl solution and EtOAc was added. The mixture was concentrated in vacuo and solid was generated, which was filtered and washed with MeOH and water to give Compound S9 (1.05 g, 43%, single diastereomer, er 93:7) as a colorless solid. This was crystallized from hexane/EtOAc (7:3 (v/v)) to give Compound S9 (590 mg, single diastereomer, er 99.7:0.3) as a colorless solid.

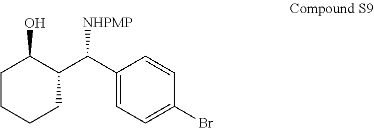

[α]²⁵_{D} −24.4 (c 0.5, EtOAc, er 99.7:0.3). ¹H NMR (500 MHz, CDCl₃): δ 7.42 (d, J=8.5 Hz, 2H), 7.22 (d, J=8.5 Hz, 2H), 6.67 (d, J=9.0 Hz, 2H), 6.50 (d, J=9.0 Hz, 2H), 4.57 (d, J=2.5 Hz, 1H), 3.68 (s, 3H), 3.41 (td, J=10.0 Hz, 4.5 Hz, 1H), 1.97-1.91 (m, 1H), 1.88-1.81 (m, 1H), 1.72-1.56 (m, 4H), 1.39-1.29 (m, 1H), 1.20-1.06 (m, 2H), 0.97 (qd, J=12.4 Hz, 3.2 Hz, 1H). ¹³C NMR (125 MHz, CDCl₃): δ 152.3, 140.5, 140.0, 131.2, 129.4, 120.6, 115.3, 114.7, 71.3, 60.8, 55.6, 49.9, 36.0, 26.9, 25.4, 24.4. HRMS (ESI): m/z calcd for C₂₀H₂₅NO₂Br [M+H]⁺ 390.1063, found 390.1048. HPLC (Daicel Chiralpak IC-3, hexane/i-PrOH=95:5, flow rate=0.5 mL/min, λ=254 nm): t_{R} 32 min (major enantiomer), 36 min (minor enantiomer).

### (Synthesis of Compound 1i)

A solution of ammonium cerium nitrate (CAN) (1.85 g, 3.4 mmol) in H₂O (9.0 mL) was added to a solution of compound S9 (300 mg, 0.77 mmol) in CH₃CN (13.5 mL) at 0° C., and the mixture was stirred at the same temperature for 2 h. The mixture was diluted with water (50 mL) and extracted with EtOAc (30 mL×3). Organic layers were combined, dried over Na₂SO₄, filtered, and concentrated. CH₂Cl₂ (10 mL) was added, and generated solid was collected by filtration to give Compound 1i (190 mg, 87%, single diastereomer, er 99.4:0.6) as a colorless solid.

A 20 mmol-scale reaction: A solution of CAN (49.48 g, 90.3 mmol) in H₂O (250 mL) to a solution of Compound S9 (8.00 g, 20.5 mmol) in CH₃CN (350 mL), was added at 0° C., and the mixture was stirred at the same temperature for 2 h. The mixture was diluted with water (250 mL), concentrated to remove the organic solvent, and extracted with EtOAc (200 mL×3). Organic layers were combined, dried over Na₂SO₄, filtered, and concentrated. CH₂Cl₂ was added, and generated solid was collected by filtration to give Compound 1i (5.61 g, 96%, single diastereomer) as a colorless solid.

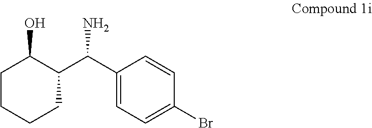 [SMILES: N[C@H](c1ccc(Br)cc1)[C@@H]1CCCC[C@H]1O]

[α]²⁵_{D} −35.7 (c 1.0, MeOH, er 99.4:0.6). ¹H NMR (400 MHz, CD₃OD): δ 7.62 (d, J=8.4 Hz, 2H), 7.38 (d, J=8.8 Hz, 2H), 4.31 (d, J=2.8 Hz, 1H), 3.24 (td, J=10.0 Hz, 4.4 Hz, 1H), 1.94-1.83 (m, 2H), 1.79-1.71 (m, 1H), 1.71-1.62 (m, 2H), 1.42-1.20 (m, 2H), 1.12-0.98 (m, 1H), 0.82 (qd, J=12.7 Hz, 3.3 Hz, 1H). ¹³C NMR (100 MHz, CD₃ OD): δ 134.5, 132.8, 131.0, 123.9, 72.0, 60.3, 47.7, 36.6, 29.2, 26.3, 25.0. HRMS (ESI): m/z calcd for C₁₃H₁₉NOBr [M+H]⁺ 284.0645, found 284.0630. HPLC (Daicel Chiralpak AS-3, hexane/i-PrOH=85:15, flow rate=1.0 mL/min, λ=220 nm): t_{R} 6.9 min (major enantiomer), 8.1 min (minor enantiomer).

### (Synthesis of Compound S10)

NaHCO₃ (5.16 g, 61.5 mmol) and di-tert-butyl decarbonate (5.64 mL, 24.6 mmol) were added to a solution of compound 1i (single diastereomer, er 99.4:0.6, 5.82 g, 20.5 mmol) in THF/H₂O (1:1 (v/v), 70 mL) at 0° C., and the mixture was stirred at room temperature (25° C.) for 12 h. The mixture was concentrated to remove the organic solvent and extracted with EtOAc (50 mL×3). Organic layers were combined, dried over Na₂SO₄, concentrated, and, dried over Na₂SO₄, concentrated, and purified by flash column chromatography (hexane/EtOAc=1:1) to give Compound S10 (6.78 g, 86%, single diastereomer) as a colorless solid.

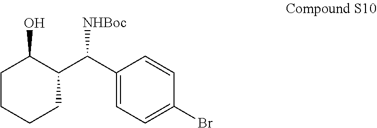 [SMILES: CC(C)(C)OC(=O)N[C@H](c1ccc(Br)cc1)[C@@H]1CCCC[C@H]1O]

¹H NMR (500 MHz, CDCl₃): δ 7.44 (d, J=8.5 Hz, 2H), 7.14 (d, J=8.5 Hz, 2H), 5.63 (d, J=8.5 Hz, 1H), 5.11-5.03 (m, 1H), 3.25 (td, J=10.0 Hz, 4.0 Hz, 1H), 2.00-1.91 (m, 1H), 1.75-1.55 (m, 3H), 1.55-1.03 (m, 4H), 1.44 (s, 9H), 0.83 (qd, J=12.5 Hz, 3.7 Hz, 1H). ¹³C NMR (125 MHz, CDCl₃): δ 156.2, 139.3, 131.2, 128.5, 120.6, 80.1, 71.1, 54.5, 50.4, 34.8, 28.3, 25.3, 24.7. HRMS (ESI): m/z calcd for C₁₋₈ H₂₇ NO₃Br [M+H]⁺ 384.1169, found 384.1184.

### (Synthesis of Compound S11)

A solution of DMSO (4.00 mL, 56.5 mmol) in CH₂Cl₂ (56 mL) was added dropwise over 20 min to a solution of oxalyl chloride (2.33 mL, 27.3 mmol) in dry CH₂Cl₂ (56 mL) at −78° C., and the mixture was stirred for 30 min at the same temperature. To the mixture, a solution of Compound S10 (7.00 g, 18.2 mmol, single diastereomer) in CH₂Cl₂ (56 mL) was added. The mixture was stirred for 30 min at the same temperature, and Et₃N (11.2 mL, 80.2 mmol) was added. The mixture was stirred for 6 h. The mixture was diluted with water (200 mL) and extracted with CH₂Cl₂. Organic layers were combined, washed with brine, dried over Na₂SO₄, filtered, concentrated, and purified by flash column chromatography (CH₂Cl₂) to give Compound S11 (4.33 g, 62%, single diastereomer, er 99.6:0.4) as a colorless solid.

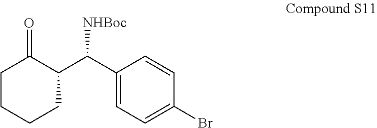 [SMILES: CC(C)(C)OC(=O)N[C@H](c1ccc(Br)cc1)[C@@H]1CCCCC1=O]

[α]^{2D} −87.1 (c 1.0, CH₂Cl₂, er 99.6:0.4). ¹H NMR (500 MHz, CDCl₃): δ 7.41-7.39 (m, 2H), 7.25-7.17 (m, 2H), 5.80 (brs, 1H), 4.82-4.73 (m, 1H), 2.88-2.76 (m, 1H), 2.38-2.20 (m, 2H), 2.15-1.79 (m, 3H), 1.78-1.49 (m, 2H), 1.44-1.23 (m, 10H). ¹³C NMR (125 MHz, CDCl₃): δ 212.5, 211.6, 155.6, 155.1, 140.8, 139.4, 131.3, 131.2, 129.9, 128.3, 121.0, 120.6, 79.6, 55.9, 54.8, 54.7, 42.6, 42.3, 32.5, 30.7, 28.2, 28.0, 26.8, 24.5, 24.4. HRMS (ESI): m/z calcd for C₁₋₈ H₂₅NO₃Br [M+H]⁺ 382.1012, found 382.1027. HPLC (Daicel Chiralpak IC-3, hexane/i-PrOH=90:10, flow rate=0.5 mL/min, λ=220 nm): t_{R} 20.7 min (minor enantiomer), 26.8 min (major enantiomer).

### (Synthesis of Compound S12)

p-Anisidine (1.66 g, 13.5 mmol) was added to a solution of compound S11 (single diastereomer, er 99.6:0.4, 4.30 g, 11.3 mmol) in 1,2-dichloroethane (120 mL) at 0° C., and the mixture was stirred for 5 minutes. To the mixture, Na(OAc)₃BH (7.15 g, 33.7 mmol) and CH₃COOH (1.93 mL, 33.7 mmol) were added, and the mixture was stirred at the same temperature for 24 h. The mixture was quenched by addition of saturated aqueous NaHCO₃ solution and extracted with CH₂Cl₂ (50 mL×3). The organic layers were combined, dried over Na₂SO₄, filtered, concentrated, and purified by flash column chromatography (hexane/EtOAc=70:30) to give the reductive amination product (5.22 g, 94%, dr 3:2) as a colorless oil. HRMS (ESI): m/z calcd for C₂₅H₃₄N₂O₃Br [M+H]⁺ 489.1747, found 489.1747.

A solution of ammonium cerium nitrate (CAN) (24.7 g, 45.0 mmol) in H₂O (140 mL) was added to a solution of the reductive amination product (5.00 g, 10.2 mmol) in CH₃CN (200 mL) at 0° C., and the mixture was stirred at the same temperature for 1 h. The mixture was diluted with water (300 mL) and extracted with EtOAc (100 mL×3). Organic layers were combined, dried over Na₂SO₄, filtered, concentrated, and purified by flash column chromatography (CH₂Cl₂/MeOH=97:3) to give Compound S12 (90 mg, 2%, single diastereomer) as a colorless oil and a mixture of Compound S12 and S12′ (3.51 g, 90%, S12/S12′=2:1). NMR data of Compound S12′ were matched to those of Compound 5. Thus, Compound S12T was the enantiomer of Compound 5. The ketone α-position of Compound S11 might be isomerized to lead to the formation of Compound S12′.

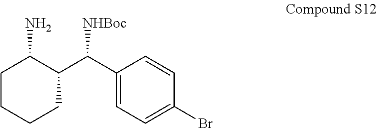 [SMILES: CC(C)(C)OC(=O)N[C@H](c1ccc(Br)cc1)[C@@H]1CCCC[C@@H]1N]

¹H NMR (500 MHz, CD₃OD): δ 7.53 (d, J=8.5 Hz, 2H), 7.32 (d, J=8.5 Hz, 2H), 4.44 (d, J=10.0 Hz, 1H), 3.13-3.08 (m, 1H), 2.12-2.05 (m, 1H), 1.98-1.81 (m, 3H), 1.68-1.29 (m, 14H). ¹³C NMR (125 MHz, CD₃OD): δ 157.9, 141.3, 133.0, 130.1, 122.6, 80.7, 56.6, 50.5, 45.4, 30.4, 28.6, 25.9, 23.0, 20.1. HRMS (ESI): m/z calcd for C₁₈H₂₈N₂O₂Br [M+H]⁺ 383.1329, found 383.1335.

### Compounds S12 and S12′ (2:1)

¹H NMR (400 MHz, CD₃OD): δ 7.57-7.49 (m, 2H), 7.32 (d, J=8.5 Hz, 2H×⅔), 7.22 (d, J=8.5 Hz, 2H×⅓), 4.44 (d, J=10 Hz, 1H×⅔), 4.28 (d, J=12.0 Hz, 1H×⅓), 3.75-3.67 (m, 1H×⅓), 3.18-3.01 (m, 1H×⅔), 2.17-0.98 (m, 19H).

### (Synthesis of Compound S13)

K₂CO₃ (4.23 g, 30.6 mmol) and 1,5-dibromopentane (4.13 mL, 30.6 mmol) were added to a solution of Compound S12 and S12′ (S12/S12′=2:1, 3.90 g, 10.2 mmol) in CH₃CN (90 mL) at room temperature (25° C.) and the mixture was refluxed at 60° C. (oil bath) for 24 h. The mixture was diluted with water and extracted with EtOAc (50 mL×3). Organic layers were combined, dried over Na₂SO₄, filtered, concentrated, and purified by flash column chromatography (CH₂Cl₂) to give Compound S13 (700 mg, 15%, single diastereomer, er 99.8:0.2) as a colorless oil.

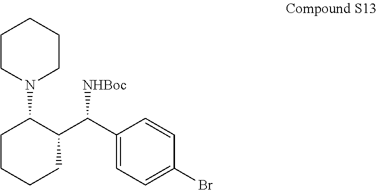 [SMILES: CC(C)(C)OC(=O)N[C@H](c1ccc(Br)cc1)[C@@H]1CCCC[C@@H]1N1CCCCC1]

[α]²⁵_{D} −33.9 (c 0.5, MeOH, er 99.8:0.2). ¹H NMR (500 MHz, CDCl₃): δ 7.38 (d, J=8.0 Hz, 2H), 7.20 (d, J=8.0 Hz, 2H), 5.46 (brs, 1H), 4.85-4.71 (m, 1H), 2.67-2.55 (m, 2H), 2.19-2.06 (m, 3H), 1.91-1.79 (m, 3H), 1.76-1.15 (m, 21H). ¹³C NMR (125 MHz, CDCl₃): δ 155.4, 142.8, 130.8, 129.3, 120.0, 79.0, 60.7, 56.9, 53.1, 45.5, 29.6, 28.3, 26.5, 25.6, 24.8, 24.4, 24.2, 23.8. HRMS (ESI): m/z calcd for C₂₃H₃₆N₂O₂Br [M+H]⁺ 451.1955, found 451.1957. HPLC (Daicel Chiralpak AS-3, hexane/i-PrOH=95:5, flow rate=1.0 mL/min, λ=220 nm): t_{R} 5.6 min (minor enantiomer), 10.6 min (major enantiomer).

(Synthesis of Compound 1g·CF₃COOH)

CF₃COOH (2 mL) was added to a solution of compound S13 (single diastereomer, er 99.8:0.2, 700 mg, 1.55 mmol) in CH₂Cl₂ (1 mL) at room temperature (25° C.), and the mixture was stirred for 2 h at the same temperature. The mixture was concentrated, and a saturated aqueous NaHCO₃ solution was added (resulting pH~7). The mixture was extracted with EtOAc (50 mL×3). Organic layers were combined, dried over Na₂SO₄, filtered, concentrated, and purified by flash column chromatography (CH₂Cl₂/MeOH=80:20) to give compound 1g·CF₃COOH (1:0.8) (290 mg, 40%, single diastereomer) as a colorless solid. The ratio between Compound 1a and CF₃COOH was determined by ¹³C NMR analysis using inverse gated ¹H decoupling mode (pulse program: zgig30, D1: 30 sec).

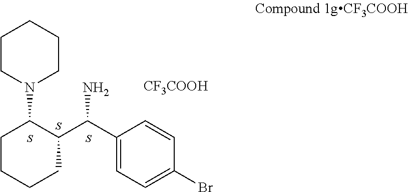

¹H NMR (500 MHz, CD₃OD): δ 7.48 (d, J=8.5 Hz, 2H), 7.31 (d, J=8.5 Hz, 2H), 4.24 (d, J=6.2 Hz, 1H), 2.99-2.88 (m, 2H), 2.77-2.73 (m, 1H), 2.57-2.51 (m, 2H), 2.14-2.07 (m, 1H), 1.95-1.80 (m, 2H), 1.67-1.22 (m, 12H). ¹³C NMR (125 MHz, CD₃ OD): δ 162.9 (q, J_{C,F}=34 Hz), 144.3, 132.2, 130.0, 121.5, 118.2 (q, J_{C,F}=291 Hz), 64.8, 58.6, 54.4, 46.8, 27.1, 26.0, 25.0, 24.5, 24.1, 23.5. HRMS (ESI): m/z calcd for C₁₈H₂₈N₂Br [M+H]⁺ 351.1430, found 351.1441.

Compound 1g·CF₃COOH was crystallized from EtOAc for X-ray crystal structural analysis. Colorless crystals, mp 180-185° C.

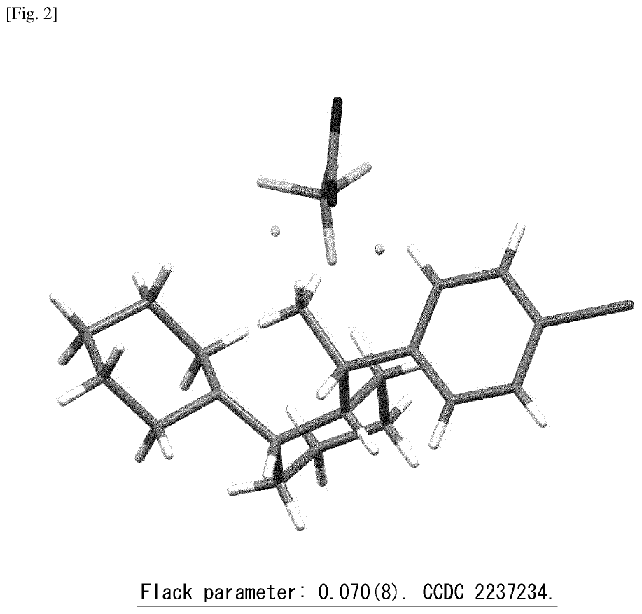

The X-ray crystal structure of Compound 1g·CF₃COOH is illustrated in FIG. 2.

### Formation of Compound 1g (Free Form)

A mixture of Compound 1g·CF₃COOH (single diastereomer, 32 mg, 0.069 mmol) and a saturated aqueous NaHCO₃ solution (15 mL) (resulting pH~8) was stirred at room temperature (25° C.) for 10 min. The mixture was extracted with CH₂Cl₂ (20 mL×3). Organic layers were combined, washed with saturated aqueous NaCl solution (15 mL), dried over Na₂SO₄, filtered, and concentrated to give Compound 1g (free form) (22 mg, 90%, single diastereomer) as a pale yellow solid.

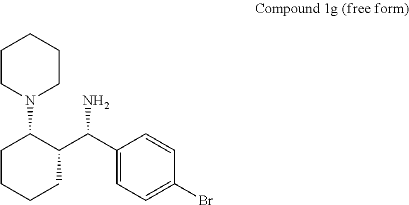 [SMILES: N[C@H](c1ccc(Br)cc1)[C@@H]1CCCC[C@@H]1N1CCCCC1]

[α]²⁵, +1132.3 (c 1.0, MeOH, er (based on S13) 99.8:0.2). ¹H NMR (500 MHz, CD₃ OD): δ 7.42 (d, J=8.4 Hz, 2H), 7.30 (d, J=8.4 Hz, 2H), 4.10 (d, J=7.5 Hz, 1H), 2.82-2.59 (m, 2H), 2.37-2.30 (m, 1H), 2.29-2.17 (m, 2H), 2.06-1.95 (m, 1H), 1.91-1.74 (m, 2H), 1.74-1.55 (m, 3H), 1.55-1.17 (m, 10H). ¹³C NMR (125 MHz, CD₃OD): S 146.3, 131.9, 130.5, 120.9, 63.3, 58.4, 54.4, 27.6, 26.1, 25.4, 25.3, 25.2, 24.9.

### Example 2 (Evaluations of Catalyst Systems)

As illustrated in following Scheme 12, we evaluated the use of 1a-l1 in the presence or absence of acids in catalysis of the Mannich reaction of β-ketophosphonate 7a with imine 8a to afford γ-position-selective Mannich reaction product 9a. The results of evaluations are illustrated in Table 1.

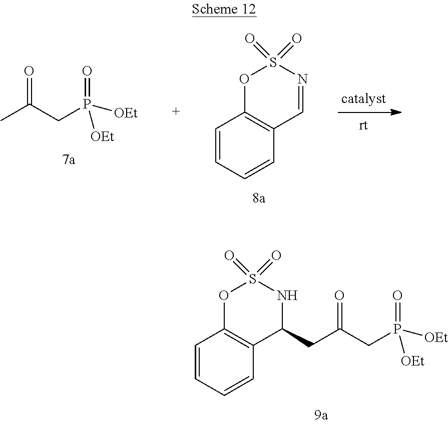 [SMILES: CCOP(=O)(CC(C)=O)OCC ; O=S1(=O)N=Cc2ccccc2O1 ; CCOP(=O)(CC(=O)C[C@@H]1NS(=O)(=O)Oc2ccccc21)OCC]

Imine 8a (0.25 mmol, 1.0 equiv) and diethyl (2-oxopropyl) phosphonate (7a) (0.75 mmol) were added to a mixture of catalyst system (as indicated, equiv relative to 8a) in toluene (1.65 mL), at room temperature (25° C.), and the mixture was stirred at the same temperature. The reaction was monitored by TLC analyses. At the indicated time, a small portion of the mixture was taken out, diluted with CDCl₃, and analyzed by ¹H NMR to determine the yield of 9a. After the ¹H NMR analysis, the remaining reaction mixture was directly purified by flash column chromatography (EtOAc) to give Compound 9a. The er of Compound 9a was determined by HPLC analysis.

TABLE 1

| | | Time | | |
|---|---|---|---|---|
| entry | Catalyst system (equiv ) | (h) | Yield (%)*1 | er *2 |
| 1 | 1a (0.3) | 96 | <5 | 82:18 |
| 2 | 1a•CF₃COOH (0.3) | 72 | 99 | 87:13 |
| 3 | 1a•CF₃COOH (0.3), | 48 | 85 | 94:6 |
| | CF₃COOH (0.3) | | | |
| 4 | 1a•CF₃COOH (0.3), | 48 | 99 | 96:4 |
| | (PhCH₂O)₃POOH (0.3) | | | |
| 5 | 1a (0.3), | 72 | 99 | 92:8 |
| | (PhCH₂O)₃POOH (0.3) | | | |
| 6 | 1a (0.3), CF₃COOH (0.3), | 48 | 99 | 96:4 |
| | (PhCH₂O)₃POOH (0.3) | | | |
| 7 | 1b•CF₃COOH (0.3), | 72 | 77 | 87:13 |
| | (PhCH₂O)₃POOH (0.3) | | | |
| 8 | 1c•CF₃COOH (0.3), | 48 | >99 | 63:37 |
| | (PhCH₂O)₃POOH (0.3) | | | |
| 9 | 1d•CF₃COOH (0.3), | 120 | 48 | 93:7 |
| | (PhCH₂O)₃POOH (0.3) | | | |
| 10 | 1e•CF₃COOH (0.3), | 48 | 89 | 94:6 |
| | (PhCH₂O)₃POOH (0.3) | | | |
| 11 | ent-1a•CF₃COOH (0.3), | 144 | 85 | 5:95 |
| | (PhCH₂O)₃POOH (0.3) | | | |
| 12 | 1g•CF₃COOH (0.3), | 96 | 80 | 16:84 |
| | (PhCH₂O)₃POOH (0.3) | | | |
| 13 | 1f (0.3), (PhCH₂O)₃POOH (0.3) | 120 | 0 | — |
| 14 | 1i (0.3), (PhCH₂O)₃POOH (0.3) | 120 | 0 | — |

*1Determined by ¹H NMR analysis before purification.
*2 Determined by HPLC analysis after purification. er:enantiomer ratio

Among the conditions tested, the reactions catalyzed by catalyst system containing 1a-1e, ent-1a and 1g afforded 9a with high enantioselectivities (Table 1, entry 1-12). The reactions where the compound was either in acid addition salt or the compound in the free form used with dibenzyl phosphate, afforded 9a with high yields (Table 1, entry 2-12). In particular, the reactions in the presence of compound 1a·CF₃COOH with dibenzyl phosphate or of compound 1a with CF₃COOH and dibenzyl phosphate at room temperature (25° C.) afforded 9a in higher yields with higher enantio selectivities (Table 1, entry 4 and 6). On the other hand, in the reactions catalyzed by catalyst system containing 1f, and 1i, the yields of 9a were 0% (Table 1, entry 13-14. Comparative examples).

### Example 3 (Evaluations of Reactions of Compounds 7 and 8 to Afford Compound 9)

As illustrated in following Scheme 13, using compound 1a·CF₃COOH with dibenzyl phosphate as the catalyst system, the scope of the γ-position-selective Mannich reactions of β-ketophosphonates was evaluated.

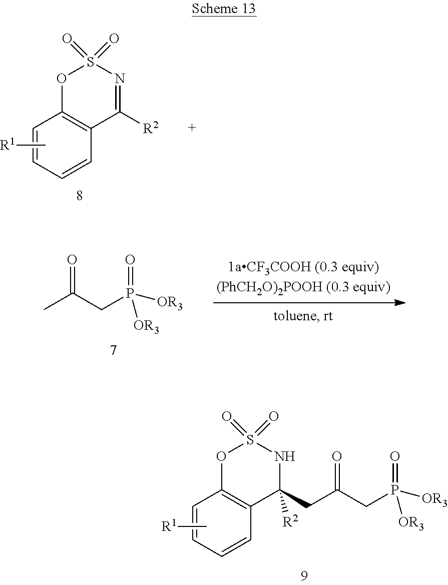

### (Synthesis of Racemic Standards of 9)

Pyrrolidine (0.1 mmol, 0.2 equiv) and CH₃COOH (0.1 mmol, 0.2 equiv) were added to a mixture of (2-oxopropyl)phosphonate 7 (1.5 mmol, 3.0 equiv) and imine 8 (0.5 mmol, 1.0 equiv) in CHCl₃ (1.0 mL) at room temperature (25° C.), and the mixture was stirred at 50° C. (oil bath) (monitored by TLC and/or ¹H NMR). After being cooled to room temperature, the mixture was directly purified by flash column chromatography (EtOAc) to give Compounds (±)-9.

### (Enantioselective Reactions of Compound 7 and Compound 8 to Afford Compound 9 Catalyzed by Compound 1a (Catalyst) and Dibenzyl Phosphate)

Imine 8 (0.25 mmol, 1.0 equiv) was added to a mixture of Compound 1a-CF₃COOH (1:1) (0.075 mmol, 0.3 equiv) and dibenzyl phosphate (0.075 mmol, 0.3 equiv) in toluene (1.65 mL) at room temperature (25° C.) followed by (2-oxopropyl)phosphonate 7 (0.75 mmol, 3.0 equiv), and the mixture was stirred at the same temperature until the imine was completely or mostly consumed (formation of 9 was monitored by ¹H NMR analyses). For the ¹H NMR analysis, a portion of the reaction mixture was taken out and diluted with CDCl₃. The ¹H NMR yield of Compound 9 was determined based on the ratio between remaining Compound 8 (imine) and Compound 9 (formed product) before purification. With the exception of the formation of Compound 9, no other products were observed to the degree that affects the determination of the yield of Compound 9. The mixture was directly purified by flash column chromatography (EtOAc) to give Compound 9. The enantiomer ratio (er) of Compound 9 was determined by HPLC analysis except Compounds 9e and 9i.

The absolute configuration of the major enantiomer of Compound 9a obtained from the reaction of Compounds 7a and 8a in the presence of Compound 1a and dibenzyl phosphate was determined to be S based on the analogy to that of Compound 11c obtained in the presence of Compound 1a and dibenzyl phosphate matched to previously reported (S)-11c. The absolute configuration of the major enantiomer of Compound 9g obtained from the reaction of Compounds 7a and 8a in the presence of Compound 1a and dibenzyl phosphate was determined to be S by X-ray crystal structural analysis. Absolute configurations of Compound 9 other than Compounds 9a and 9g were determined by analogy.

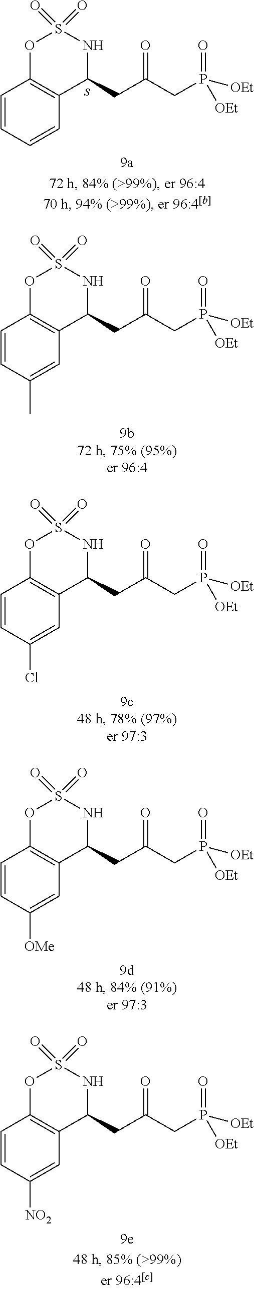

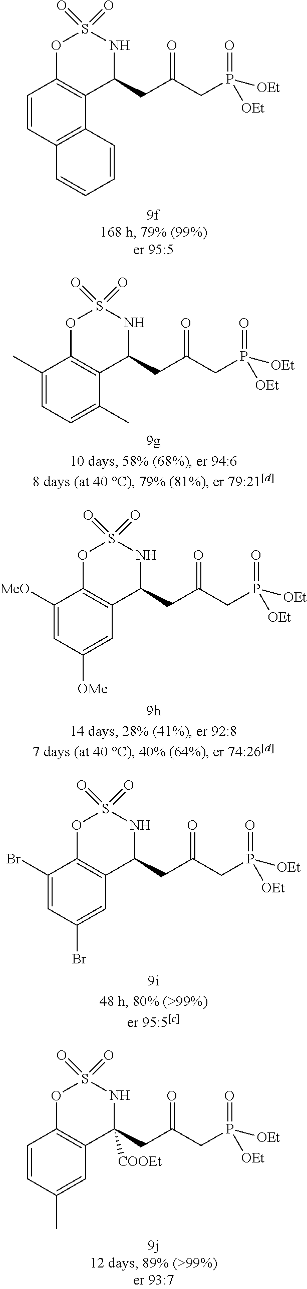 [SMILES: CCOP(=O)(CC(=O)C[C@@H]1NS(=O)(=O)Oc2ccc3ccccc3c21)OCC ; CCOP(=O)(CC(=O)C[C@@H]1NS(=O)(=O)Oc2c(C)ccc(C)c21)OCC ; CCOP(=O)(CC(=O)C[C@@H]1NS(=O)(=O)Oc2c(OC)cc(OC)cc21)OCC ; CCOP(=O)(CC(=O)C[C@@H]1NS(=O)(=O)Oc2c(Br)cc(Br)cc21)OCC ; CCOC(=O)[C@@]1(CC(=O)CP(=O)(OCC)OCC)NS(=O)(=O)Oc2ccc(C)cc21]

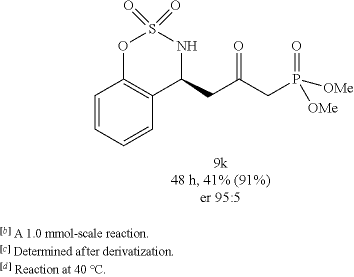 [SMILES: COP(=O)(CC(=O)C[C@@H]1NS(=O)(=O)Oc2ccccc21)OC]

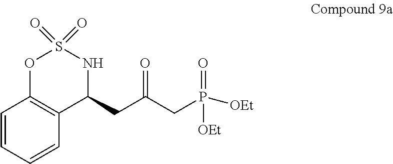 [SMILES: CCOP(=O)(CC(=O)C[C@@H]1NS(=O)(=O)Oc2ccccc21)OCC]

General procedure, 72 h, flash column chromatography (EtOAc), 78.8 mg (84%, er 96:4), colorless solid.

[α]_{D}²⁴ −30.4 (c=1.0, MeOH, er 96:4). Lit. [α]_{D}²⁴ −31.9 (c=1.0, THF, er 94:6). ¹H and ¹³C NMR data were in agreement with previously reported data. ¹H NMR (500 MHz, CDCl₃) δ 7.32-7.29 (m, 1H), 7.20-7.15 (m, 2H), 7.02 (d, J=8.1 Hz, 2H), 6.21 (d, J=8.1 Hz, 1H), 5.22 (td, J=8.1 Hz, 3.8 Hz, 1H), 4.17-4.09 (m, 4H), 3.79 (dd, J=18.0 Hz, 8.1 Hz, 1H), 3.22-3.10 (m, 3H), 1.33 (t, J=7.1 Hz, 3H), 1.31 (t, J=7.1 Hz, 3H). ¹³C NMR (125 MHz, CDCl₃): δ 199.80, 199.75, 151.2, 129.6, 126.0, 125.3, 121.2, 119.0, 63.23, 63.17, 63.01, 62.96, 53.2, 46.9, 43.9, 42.9, 16.28, 16.25, 16.24, 16.20. HRMS (ESI): m/z calcd for C₁₄H₂₁O₇NPS [M+H]⁺ 378.0771, found 378.0769. HPLC (Daicel Chiralpak IC-3, hexane/i-PrOH=80:20, flow rate=1.0 mL/min, λ=220 nm): t_{R} (minor enantiomer)=56 min, t_{R} (major enantiomer)=80 min.

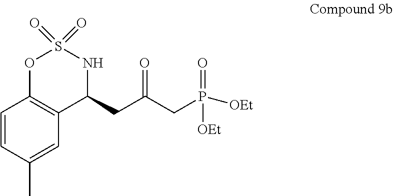 [SMILES: CCOP(=O)(CC(=O)C[C@@H]1NS(=O)(=O)Oc2ccc(C)cc21)OCC]

General procedure, 72 h, flash column chromatography (EtOAc), 73.2 mg (75%, er 96:4), colorless oil.

[α]_{D}²⁴ −30.5 (c=1.0, MeOH, er 96:4). ¹H NMR (500 MHz, CDCl₃) δ 7.09 (d, J=8.5 Hz, 1H), 6.96 (s, 1H), 6.90 (d, J=8.5 Hz, 1H), 6.04 (d, J=8.3 Hz, 1H), 5.17 Hz (td, J=8.3 Hz, 3.5 Hz, 1H), 4.19-4.08 (m, 4H), 3.77 (dd, J=18.1 Hz, 8.3 Hz, 1H), 3.22-3.09 (m, 3H), 2.30 (s, 3H), 1.33 (t, J=7.3 Hz, 3H), 1.31 (t, J=7.3 Hz, 3H). ¹³C NMR (125 MHz, CDCl₃): δ 199.81, 199.76, 149.1, 135.1, 130.2, 126.3, 120.7, 118.7, 63.2, 63.1, 63.0, 62.9, 53.2, 47.0, 43.9, 42.9, 25.3, 20.8, 16.28, 16.26, 16.23, 16.21. HRMS (ESI): m/z calcd for C₁₅H₂₃O₇NPS [M+H]⁺ 392.0927, found 392.0921. HPLC (Daicel Chiralpak IC-3, hexane/i-PrOH=80:20, flow rate=1.0 mL/min, λ=220 nm): t_{R} (minor enantiomer)=56 min, t_{R} (major enantiomer)=95 min.

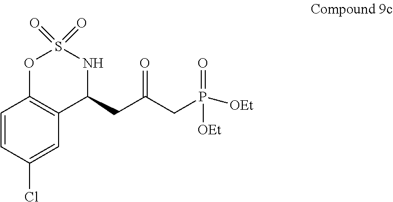 [SMILES: CCOP(=O)(CC(=O)C[C@@H]1NS(=O)(=O)Oc2ccc(Cl)cc21)OCC]

General procedure, 48 h, flash column chromatography (EtOAc), 80.7 mg (78%, er 97:3), colorless solid.

[α]_{D}²⁴ −35.4 (c=1.0, MeOH, er 97:3). ¹H NMR (500 MHz, CDCl₃) δ 7.27-7.24 (m, 1H), 7.19-7.17 (m, 1H), 6.95 (d, J=8.5 Hz, 1H), 6.62 (d, J=8.0 Hz, 1H), 5.18 (td, J=8.0 Hz, 4.0 Hz, 1H), 4.17-4.08 (m, 4H), 3.77 (dd, J=18.3 Hz, 8.0 Hz, 1H), 3.24-3.09 (m, 3H), 1.31 (t, J=7.1 Hz, 3H), 1.30 (t, J=7.1 Hz, 3H). ¹³C NMR (125 MHz, CDCl₃): δ 199.42, 199.38, 149.7, 130.4, 129.6, 126.1, 123.0, 120.3, 63.3, 63.2, 63.1, 63.0, 52.7, 46.9, 43.6, 42.6, 16.23, 16.22, 16.19, 16.18. HRMS (ESI): m/z calcd for C₁₄H₂₀O₇ NClPS [M+H]⁺ 412.0381, found 412.0357. HPLC (Daicel Chiralpak IC-3, hexane/i-PrOH=80:20, flow rate=1.0 mL/min, λ=220 nm): t_{R} (minor enantiomer)=36 min, t_{R} (major enantiomer)=56 min.

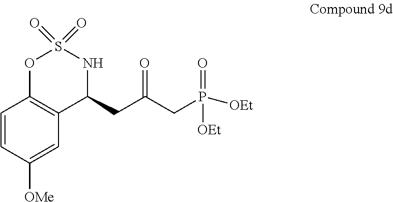 [SMILES: CCOP(=O)(CC(=O)C[C@@H]1NS(=O)(=O)Oc2ccc(OC)cc21)OCC]

General Procedure, 48 h, flash column chromatography (EtOAc), 85.9 mg (84%, er 97:3), white solid.

[α]_{D}²⁴ −40.4 (c=1.0, MeOH, er 97:3). ¹H NMR (500 MHz, CDCl₃) δ 6.94 (d, J=9.0 Hz, 1H), 6.82 (dd, J=9.0 Hz, 2.7 Hz, 1H), 6.68 (d, J=2.7 Hz, 1H), 6.24 (d, J=8.4 Hz, 1H), 5.16 (td, J=8.4 Hz, 3.9 Hz, 1H), 4.17-4.07 (m, 4H), 3.78 (dd, J=18.3 Hz, 8.4 Hz, 1H), 3.77 (s, 3H), 3.21-3.09 (m, 3H), 1.31 (t, J=7.0 Hz, 3H), 1.29 (t, J=7.0 Hz, 3H). ¹³C NMR (125 MHz, CDCl₃): δ 199.81, 199.76, 156.7, 144.9, 122.0, 119.9, 114.8, 110.9, 63.2, 63.1, 63.0, 62.9, 55.7, 53.2, 47.0, 43.7, 42.7, 16.24, 16.19, 16.15. HRMS (ESI): m/z calcd for C₁₅H₂₃O₈NPS [M+H]⁺ 408.0877, found 408.0875. HPLC (Daicel Chiralpak IC-3, hexane/i-PrOH=80:20, flow rate=1.0 mL/min, λ=220 nm): t_{R} (minor enantiomer)=69 min, t_{R} (major enantiomer)=106 min.

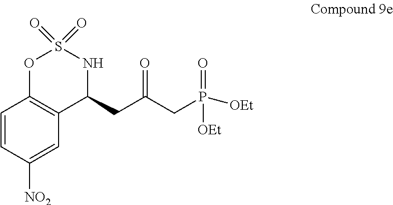 [SMILES: CCOP(=O)(CC(=O)C[C@@H]1NS(=O)(=O)Oc2ccc([N+](=O)[O-])cc21)OCC]

General procedure, 48 h, flash column chromatography (EtOAc), 89.9 mg (85%, er (determined after transformation to 9e-OH) 96:4), colorless oil. ¹H NMR (500 MHz, CDCl₃) δ 8.20 (dd, J=9.0 Hz, 2.5 Hz, 1H), 8.13 (d, J=2.5 Hz, 1H), 7.18 (d, J=9.0 Hz, 1H), 6.79 (d, J=8.6 Hz, 1H), 5.33-5.27 (m, 1H), 4.19-4.10 (m, 4H), 3.85 (dd, J=18.2 Hz, 7.8 Hz, 1H), 3.28-3.12 (m, 3H), 1.33 (t, J=7.2 Hz, 3H), 1.32 (t, J=7.2 Hz, 3H). ¹³C NMR (125 MHz, CDCl₃): δ 199.01, 198.97, 155.6, 144.4, 125.0, 122.7, 122.3, 120.1, 63.42, 63.37, 63.22, 63.16, 52.7, 46.8, 43.7, 42.7, 16.24, 16.22, 16.19, 16.17. HRMS (ESI): m/z calcd for C₁₄H₂₀O₉N₂PS [M+H]⁺ 423.0622, found 424.0668.

(Transformation of Compound 9e-OH to Compound 9e)

NaBH₄ (38 mg, 0.10 mmol) was added to a solution of Compound 9e (42.2 mg, 0.1 mmol in MeOH (200 μL) at 0° C., and the mixture was stirred for 45 min at the same temperature. The mixture was purified by preparative TLC (EtOAc) to afford Compound 9e-OH (7.0 mg, 17%, dr 2:1, a mixture of the diastereomers was isolated) as a colorless oil.

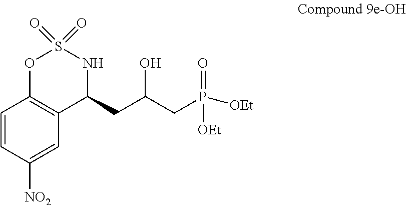 [SMILES: CCOP(=O)(CC(O)C[C@@H]1NS(=O)(=O)Oc2ccc([N+](=O)[O-])cc21)OCC]

A mixture of two diastereomers, dr 2:1, er major diastereomer 93:7, er minor diastereomer 96:4. The higher er value was used for the er of Compound 9e as isomerization of Compound 9e may occur under the conditions used in the formation of Compound 9e-OH.

¹H NMR (500 MHz, CDCl₃) δ 8.22 (dd, J=9.0 Hz, 2.0 Hz, 1H×⅔), 8.20 (dd, J=9.0 Hz, 2.0 Hz, 1H×⅓), 8.17 (d, J=2.0 Hz, 1H×⅓), 8.12-8.10 (m, 1H×⅔), 7.20 (d, J=9.0 Hz, 1H×⅔), 7.17 (d, J=9.0 Hz, 1H×⅓), 6.96-6.88 (m, 1H), 5.15 (td, J=8.0 Hz, 2.5 Hz, 1H×⅔), 5.13-5.07 (m, 1H×⅓), 4.43-4.35 (m, 1H×⅓), 4.25-4.01 (m, 1H×⅔+4H), 2.49-1.95 (m, 4H), 1.39-1.22 (m, 6H). ¹³C NMR (125 MHz, CDCl₃): δ 156.1, 155.2, 144.42, 144.38, 124.94, 124.88, 123.8, 122.7, 122.4, 122.3, 120.4, 120.2, 65.20, 65.17, 63.39, 63.35, 62.53, 62.50, 62.48, 62.45, 62.4, 62.3, 55.21, 55.20, 55.11, 55.09, 41.3, 41.2, 39.5, 39.3, 34.0, 33.9, 32.9, 32.8, 16.37, 16.34, 16.31, 16.29, 16.24. HRMS (ESI): m/z calcd for C₁₄H₂₂O₉N₂PS [M+H]⁺ 425.0778, found 425.0782. HPLC (Daicel Chiralpak IC-3, hexane/i-PrOH=85:15, flow rate=0.8 mL/min, λ=220 nm): t_{R} (major diastereomer, minor enantiomer)=50 min, t_{R}(major diastereomer, major enantiomer)=82 min, t_{R} (minor diastereomer, minor enantiomer)=74 min, t_{R} (minor diastereomer, major enantiomer)=109 min.

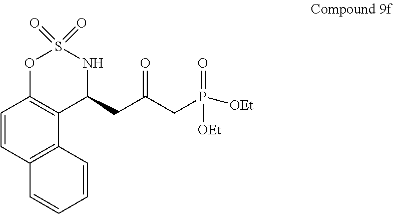 [SMILES: CCOP(=O)(CC(=O)C[C@@H]1NS(=O)(=O)Oc2ccc3ccccc3c21)OCC]

General procedure, 168 h, flash column chromatography (EtOAc), 84.5 mg (79%, er 95:5), white solid.

[α]_{D}²⁴ −44.1 (c=1.0, MeOH, er 95:5). ¹H NMR (500 MHz, CDCl₃) δ 7.83 (d, J=8.3 Hz, 1H), 7.78 (d, J=8.9 Hz, 1H), 7.59 (d, J=8.3 Hz, 1H), 7.56-7.51 (m, 1H), 7.49-7.45 (m, 1H), 7.09 (d, J=8.9 Hz, 1H), 6.50 (d, J=6.3 Hz, 1H), 5.81 (ddd, J=10.6 Hz, 6.3 Hz, 2.0 Hz, 1H), 4.16-4.06 (m, 4H), 4.00 (dd, J=18.7 Hz, 10.6 Hz, 1H), 3.24-3.11 (m, 2H), 3.08 (dd, J=18.7 Hz, 2.0 Hz, 1H), 1.26 (t, J=7.1 Hz, 6H). ¹³C NMR (125 MHz, CDCl₃): δ 199.7, 199.6, 149.1, 131.1, 130.7, 129.21, 129.18, 128.0, 125.7, 122.0, 118.4, 114.0, 62.93, 62.88, 62.85, 62.79, 51.4, 47.0, 43.2, 42.2, 16.22, 16.20, 16.18, 16.15. HRMS (ESI): m/z calcd for C₁₋₈ H₂₃O₇NPS [M+H]⁺ 428.0927, found 428.0922. HPLC (Daicel Chiralpak IC-3, hexane/i-PrOH=80:20, flow rate=0.8 mL/min, λ=220 nm): t_{R} (minor enantiomer)=61 min, t_{R} (major enantiomer)=90 min.

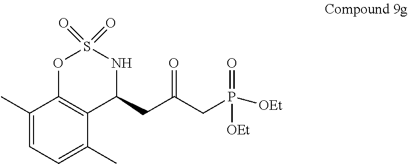 [SMILES: CCOP(=O)(CC(=O)C[C@@H]1NS(=O)(=O)Oc2c(C)ccc(C)c21)OCC]

General procedure (imine not fully consumed), 10 days (¹H NMR yield 68%), flash column chromatography (EtOAc), 59.0 mg (58%, er 94:6), colorless solid.

[α]_{D}²⁴ −14.7 (c=1.0, MeOH, er 94:6). ¹H NMR (500 MHz, CDCl₃) δ 7.08 (d, J=7.8 Hz, 1H), 6.92 (d, J=7.8 Hz, 1H), 5.52 (d, J=5.6 Hz, 1H), 5.25 (ddd, J=10.3 Hz, 5.6 Hz, 2.5 Hz, 1H), 4.20-4.10 (m, 4H), 3.86 (dd, J=18.5 Hz, 10.3 Hz, 1H), 3.23 (dd, J=22.5 Hz, 13.5 Hz, 1H), 3.13 (dd, J=22.5 Hz, 13.5 Hz, 1H), 2.93 (dd, J=18.5 Hz, 2.5 Hz, 1H), 2.24 (s, 3H), 2.23 (s, 3H), 1.33 (t, J=7.1 Hz, 6H). ¹³C NMR (125 MHz, CDCl₃): δ 199.40, 199.35, 150.0, 132.5, 130.7, 127.1, 125.9, 120.2, 62.93, 62.88, 62.82, 62.77, 51.7, 46.4, 43.5, 42.5, 18.6, 16.31, 16.29, 16.27, 16.24, 15.3. HRMS (ESI): m/z calcd for C₁₆H₂₅O₇NPS [M+H]⁺ 406.1084, found 406.1092. HPLC (Daicel Chiralpak IC-3, hexane/i-PrOH=80:20, flow rate=1.0 mL/min, λ=220 nm): t_{R}(minor enantiomer)=62 min, t_{R} (major enantiomer)=71 min.

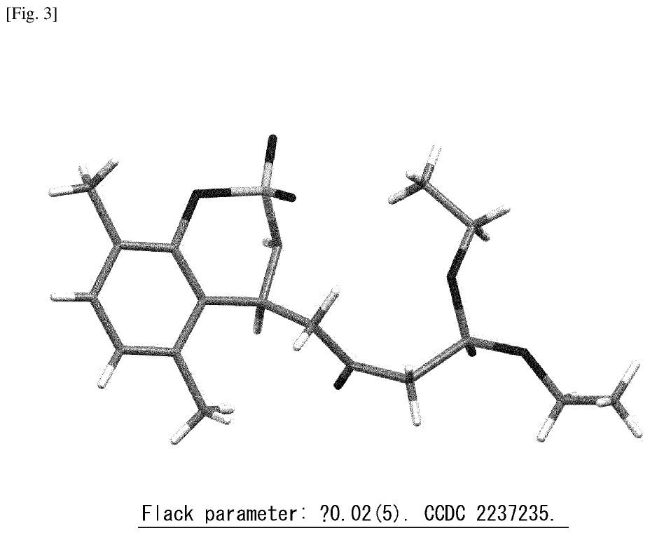

Compound 9g was crystallized from CHCl₃. Colorless crystals, mp 95-100° C., er 96:4. HPLC (Daicel Chiralpak IC-3, hexane/i-PrOH=80:20, flow rate=1.0 mL/min, λ=220 nm): t_{R} (minor enantiomer)=64 min, t_{R} (major enantiomer)=75 min. The X-ray crystal structure of compound 9g is illustrated in FIG. 3.

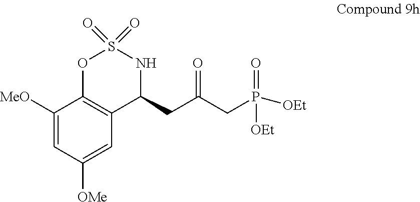 [SMILES: CCOP(=O)(CC(=O)C[C@@H]1NS(=O)(=O)Oc2c(OC)cc(OC)cc21)OCC]

General procedure (imine not fully consumed), 14 days (¹H NMR yield 41%), flash column chromatography (EtOAc), 30.1 mg (28%, er 92:8), colorless oil.

[α]_{D}²⁴ −20.0 (c=1.0, MeOH, er 92:8). ¹H NMR (500 MHz, CDCl₃) δ 6.26 (d, J=2.4 Hz, 1H), 6.16 (d, J=2.4 Hz, 1H), 5.63 (d, J=6.5 Hz, 1H), 5.21 (ddd, J=10.1 Hz, 6.5 Hz, 2.5 Hz, 1H), 4.20-4.08 (m, 4H), 3.80 (s, 6H), 3.78 (s, 3H), 3.66 (dd, J=17.8 Hz, 10.1 Hz, 1H), 3.22-3.07 (m, 3H), 1.344 (t, J=7.0 Hz, 3H), 1.339 (t, J=7.0 Hz, 3H). ¹³C NMR (125 MHz, CDCl₃): δ 200.14, 200.09, 161.0, 156.9, 152.3, 102.1, 95.8, 95.2, 62.9, 62.82, 62.80, 62.75, 55.9, 55.6, 50.6, 46.3, 43.6, 42.6, 16.32, 16.30, 16.27, 16.25. HRMS (ESI): m/z calcd for C₁₆H₂₅O₉NPS [M+H]⁺ 438.0982, found 438.1030. HPLC (Daicel Chiralpak IC-3, hexane/i-PrOH=70:30, flow rate=1.0 mL/min, λ=220 nm): t_{R} (minor enantiomer)=49 min, t_{R} (major enantiomer)=70 min.

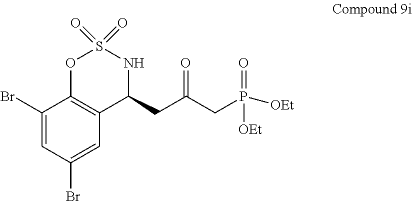 [SMILES: CCOP(=O)(CC(=O)C[C@@H]1NS(=O)(=O)Oc2c(Br)cc(Br)cc21)OCC]

General procedure, 48 h, flash column chromatography (EtOAc), 106.9 mg (80%, er (determined after transformation to 9i-OH) 95:5), colorless oil.

¹H NMR (500 MHz, CDCl₃) δ 7.68-7.67 (m, 1H), 7.29-7.28 (m, 1H), 6.97 (d, J=8.4 Hz, 1H), 5.23-5.17 (m, 1H), 4.16-4.08 (m, 4H), 3.76 (dd, J=18.3 Hz, 7.8 Hz, 1H), 3.24-3.09 (m, 3H), 1.31 (t, J=7.0 Hz, 6H). ¹³C NMR (125 MHz, CDCl₃): δ 199.1, 199.0, 147.3, 135.6, 128.2, 125.0, 117.7, 113.6, 63.4, 63.3, 63.2, 63.1, 52.7, 47.1, 43.5, 42.5, 16.26, 16.25, 16.22, 16.20. HRMS (ESI): m/z calcd for C₁₄H₁₉O₇NBr₂PS [M+H]⁺ 533.8981, found 533.9040.

(Transformation of Compounds 9i to 9i-OH)

NaBH₄ (38 mg, 0.1 mmol) was added to a solution of Compound 9i (53.5 mg, 0.1 mmol in MeOH (200 μL), at 0° C., and the mixture was stirred for 45 min at the same temperature. The mixture was purified by preparative TLC (EtOAc) to afford Compound 9i-OH (11 mg, 21%, dr 2:1, a mixture of the diastereomers was isolated) as a colorless oil.

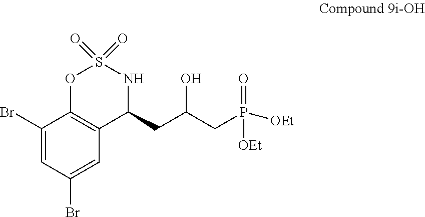 [SMILES: CCOP(=O)(CC(O)C[C@@H]1NS(=O)(=O)Oc2c(Br)cc(Br)cc21)OCC]

A mixture of two diastereomers, dr 2:1, er major diastereomer 95:5, er minor diastereomer 95:5.

¹H NMR (500 MHz, CDCl₃): δ 7.71-7.69 (m, 1H), 7.32-7.30 (m, 1H×⅓), 7.26-7.23 (m, 1H×⅔), 6.87 (d, J=8.4 Hz, 1H×⅔), 6.80 (d, J=6.0 Hz, 1H×⅓), 5.06-5.02 (m, 1H×⅔), 4.99-4.95 (m, 1H×⅓), 4.35-4.02 (m, 5H), 2.38-1.92 (m, 5H), 1.37-1.30 (m, 6H). ¹³C NMR (125 MHz, CDCl₃): δ 147.9, 147.2, 135.52, 135.46, 128.6, 128.2, 126.2, 124.8, 117.7, 117.6, 114.0, 113.8, 65.02, 64.98, 63.33, 63.29, 62.47, 62.42, 62.40, 62.35, 55.20, 55.17, 55.1, 41.6, 41.4, 40.0, 39.9, 34.1, 33.8, 33.0, 32.7, 16.4, 16.3. HRMS (ESI): m/z calcd for C₁₄H₂₁O₇NBr₂PS [M+H]⁺ 535.9138, found 535.9178. HPLC (Daicel Chiralpak IC-3, hexane/i-PrOH=92:8, flow rate=0.8 mL/min, λ=220 nm): t_{R} (major diastereomer, minor enantiomer)=79 min, t_{R} (major diastereomer, major enantiomer)=84 min, t_{R} (minor diastereomer, minor enantiomer)=106 min, t_{R} (minor diastereomer, major enantiomer)=115 min.

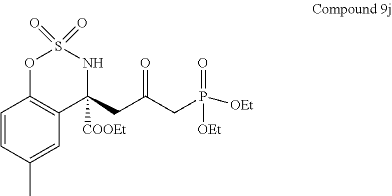 [SMILES: CCOC(=O)[C@@]1(CC(=O)CP(=O)(OCC)OCC)NS(=O)(=O)Oc2ccc(C)cc21]

General procedure, 12 days, flash column chromatography (EtOAc), 103.0 mg (89%, er 93:7), colorless solid.

[α]_{D}²⁴ −29.0 (c=1.0, MeOH, er 93:7). Lit. [α]_{D}²⁴ −51.4 (c=1.0, CHCl₃, er 95.7:4.3). ¹H and ¹³C NMR data were in agreement with previously reported data in V. Bethi, F. Tanaka, Org. Lett. 2022, 24,6711.

HRMS (ESI): m/z calcd for C₁₋₈ H₂₇O₉NPS [M+H]⁺ 464.1139, found 464.1138. HPLC (Daicel Chiralpak IF-3, hexane/i-PrOH=80:20, flow rate=1.0 mL/min, λ=220 nm): t_{R} (major enantiomer)=35 min, t_{R} (minor enantiomer)=51 min.

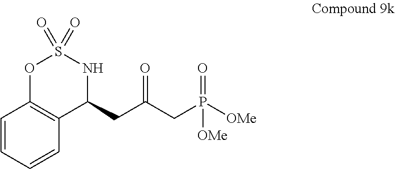 [SMILES: COP(=O)(CC(=O)C[C@@H]1NS(=O)(=O)Oc2ccccc21)OC]

General procedure, 48 h, flash column chromatography (EtOAc), 35.9 mg (41%, er 95:5), white solid.

[α]_{D}²⁴ −18.3 (c=1.0, MeOH, er 95:5). ¹H NMR (500 MHz, CDCl₃) δ 7.35-7.29 (m, 1H), 7.20-7.16 (m, 2H), 7.04 (d, J=8.1 Hz, 1H), 5.90 (d, J=8.3 Hz, 1H), 5.22 (td, J=8.3 Hz, 3.7 Hz, 1H), 3.82 (dd, J=18.0 Hz, 8.3 Hz, 1H), 3.80 (d, J=12.0 Hz, 3H), 3.78 (d, J=12.0 Hz, 3H), 3.20 (dd, J=23.0 Hz, 13.5 Hz, 1H), 3.15 (dd, J=23.0 Hz, 13.3 Hz, 1H), 3.14 (dd, J=18.0 Hz, 3.7 Hz, 1H). ¹³C NMR (125 MHz, CDCl₃): δ 199.63, 199.58, 151.2, 129.7, 126.0, 125.4, 120.9, 119.2, 53.48, 53.43, 53.28, 53.24, 46.8, 42.9, 41.9. HRMS (ESI): m/z calcd for C₁₂H₁₇O₇NPS [M+H]⁺ 350.0458, found 350.0490. HPLC (Daicel Chiralpak IC-3, hexane/i-PrOH=80:20, flow rate=0.8 mL/min, λ=220 nm): t_{R} (minor enantiomer)=28 min, t_{R} (major enantiomer)=40 min.

### Example 4 (Evaluations of Mannich Reactions of β-Ketocarbonyl Compounds in the Presence of the Catalyst System Containing Compound 1a)

To further understand the features of the catalysis by Compound 1a, reactions of β-ketocarbonyl compounds other than β-ketophosphonates with Compound 8a (imine) under the conditions used for the formation of Compound 9 were evaluated. The reaction is illustrated in Scheme 14.

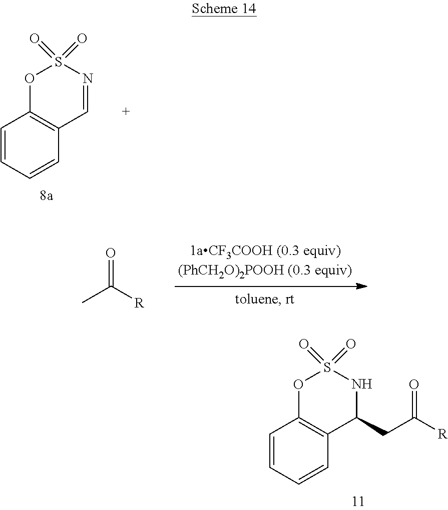

### (Synthesis of Racemic Standards of 11)

Racemic standards of Compound 11 were synthesized using pyrrolidine-acetic acid (1:1) as catalyst by the reported procedures as the same procedure to form (±)-9 as described above.

### (General Procedure of the Enantioselective Reactions of Ketones and Compound 8a to Afford Compound 11 in the Presence of Compound 1a and Dibenzyl Phosphate)

Compound 8a (0.25 mmol, 1.0 equiv) was added to a mixture of Compound 1a-CF₃COOH (1:1) (0.075 mmol, 0.3 equiv) and dibenzyl phosphate (0.075 mmol, 0.3 equiv) in toluene (1.65 mL), at room temperature (25° C.) followed by ketone (CH₃(═O)R) (0.75 mmol, 3.0 equiv), and the mixture was stirred at the same temperature until the imine was completely or mostly consumed (formation of 11 was monitored by ¹H NMR analyses). For the ¹H NMR analysis, a portion of the reaction mixture was taken out and diluted with CDCl₃. The ¹H NMR yield of Compound 11 was determined based on the ratio among remaining imine 8a, γ-position reaction product 11, and the α-position reaction product (if any) before purification. The mixture was directly purified by flash column chromatography to give Compound 11. For Compound 11a, enantiomer ratio (er) was determined after transformation to derivative 11a-NOH. For Compounds 11b and 11c, the er was determined by HPLC analysis.

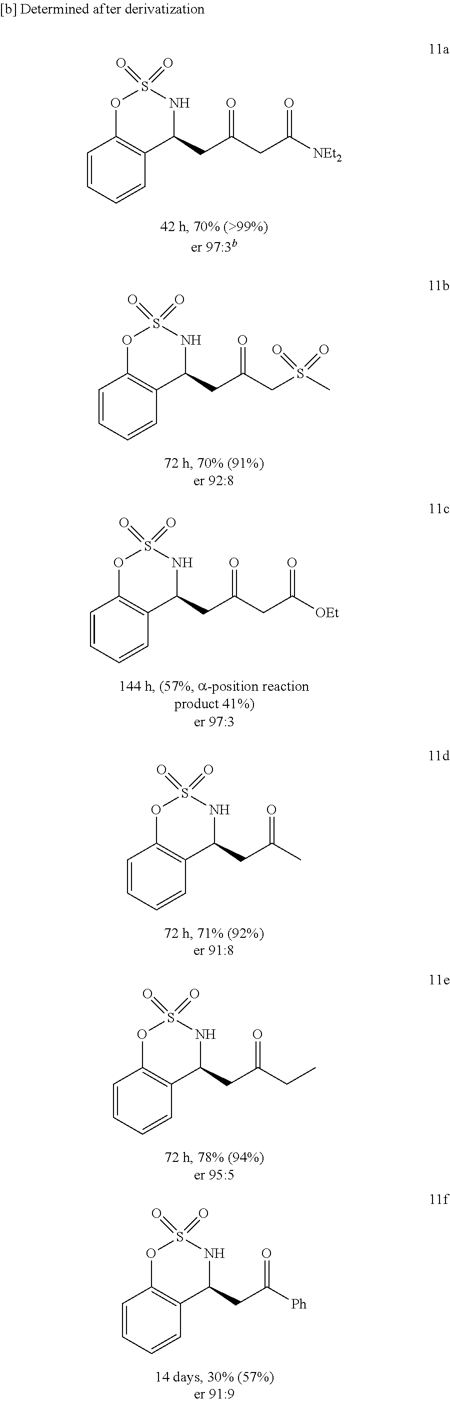

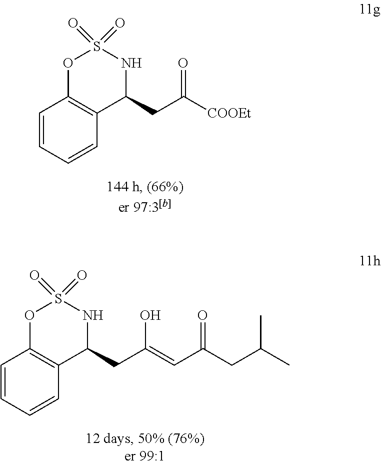 [SMILES: CCOC(=O)C(=O)C[C@@H]1NS(=O)(=O)Oc2ccccc21 ; CC(C)CC(=O)/C=C(\O)C[C@@H]1NS(=O)(=O)Oc2ccccc21]

In the reactions of β-ketocarbonyl compounds other than β-ketophosphonates with Compound 8a (imine) under the conditions used for the formation of Compound 9, γ-position reaction products which are Compounds 11a-11h, respectively, were obtained, and no formation of α-position reaction products was observed.

### Compound 11a

General procedure, 48 h, flash column chromatography (CH₂Cl₂/EtOAc=90:10), 59.2 mg (70%, keto form/enol form=2:1, er (determined after transformation to 11a-NOH) 97:3).

¹H and ¹³C NMR data were in agreement with previously reported data in V. Bethi, F. Tanaka, Org. Lett. 2022, 24,6711.

(Transformation of Compound 11a to Compound 11a-NOH)

Compound 11a (35.0 mg, 0.10 mmol) was transformed to Compound 11a-NOH by the reported procedure in V. Bethi. F. Tanaka. Org. Lett. 2022. 24.6711. 11a-NOH (23.3 mg, 83%, er 97:3) was obtained as a colorless solid.

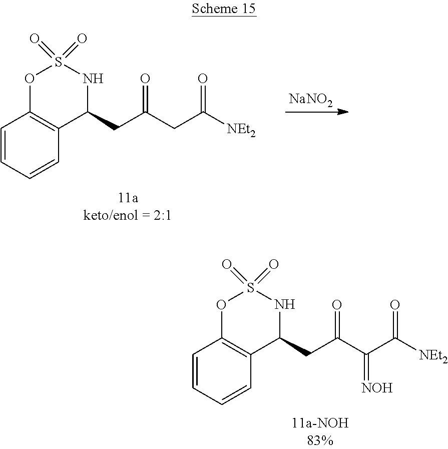

Compound 11a-NOH

[α]_{D}²⁴ −30.0 (c=0.4, MeOH, er 97:3). Lit. [α]_{D}²⁴ −2.1 (c=1.0, CHCl₃, er 95.6:4.4). ¹H and ¹³C NMR data were in agreement with previously reported data in V. Bethi, F. Tanaka, Org. Lett. 2022, 24,6711. HPLC (Daicel Chiralpak IC-3, hexane/i-PrOH=80:20, flow rate=0.8 mL/min, λ=220 nm): t_{R} (minor enantiomer)=33 min, t_{R} (major enantiomer)=46 min.

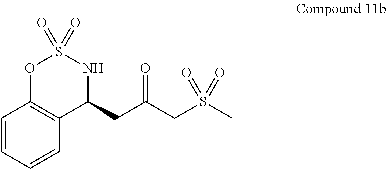 [SMILES: CS(=O)(=O)CC(=O)C[C@@H]1NS(=O)(=O)Oc2ccccc21]

General procedure, 72 h, flash column chromatography (CH₂Cl₂/EtOAc=90:10), 58.8 mg (70%, er 92:8), colorless solid.

¹H NMR (500 MHz, (CD₃)₂SO): δ 8.73 (s, 1H), 7.40-7.32 (m, 2H), 7.28-7.22 (m, 1H), 7.11-7.09 (m, 1H), 5.15 (dd, J=8.8 Hz, 3.8 Hz, 1H), 4.63 (d, J=15.0 Hz, 1H), 4.59 (d, J=15.0 Hz, 1H), 3.47 (dd, J=18.5 Hz, 8.8 Hz, 1H), 3.35 (dd, J=18.5 Hz, 3.8 Hz, 1H), 3.12 (s, 3H). ¹³C NMR (125 MHz, (CD₃)₂SO): δ 198.1, 151.6, 130.5, 128.1, 126.2, 122.9, 119.2, 64.7, 52.2, 49.5, 42.8. HPLC (Daicel Chiralpak IC-3, hexane/i-PrOH=70:30, flow rate=0.8 mL/min, λ=220 nm): t_{R} (minor enantiomer)=51 min, t_{R} (major enantiomer)=74 min.

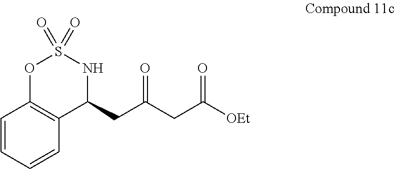 [SMILES: CCOC(=O)CC(=O)C[C@@H]1NS(=O)(=O)Oc2ccccc21]

General procedure, 144 h, ¹H NMR yield of 11c 57%, ¹H NMR yield of the corresponding α-position reaction product 41%. Compound 11c was purified by preparative TLC (hexane/EtOAc=80/20), er 97:3, colorless oil.

[α]D²⁴ −19.5 (c=0.4, MeOH, er 97:3). Lit. [α]D²⁴ −59.3 (c=1.0, CHCl₃, er 98.9:1.1). ¹H and ¹³C NMR data of Compound 11c were in agreement with previously reported data in V. Bethi, F. Tanaka, Org. Lett. 2022, 24,6711. HPLC (Daicel Chiralpak IC-3, hexane/i-PrOH=80:20, flow rate=0.8 mL/min, λ=220 nm): t_{R} (major enantiomer)=32 min, t_{R} (minor enantiomer)=43 min.

It was confirmed that the target compounds were obtained based on ¹H and ¹³C NMR data of Compounds 11d to 11h.

### Example 4 (Evaluation of Mannich-Aza-Michael Reaction to Afford Compound 12)

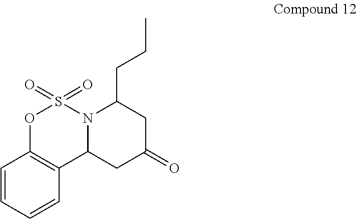 [SMILES: CCCC1CC(=O)CC2c3ccccc3OS(=O)(=O)N12]

The Mannich-aza-Michael reaction shown below was performed using the catalyst system containing 1a. The reaction product (Compound 12) is known (Org. Lett. 2013, 15, 6090; other catalysts were used to form this product with dr>19:1, 95% ee).

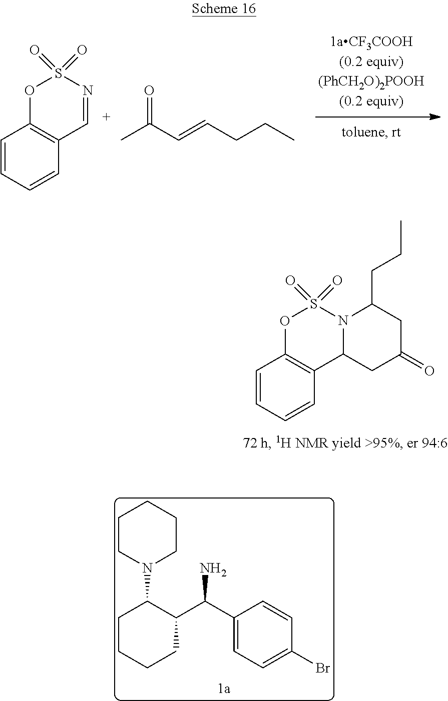

To a mixture of 1a-CF₃COOH (1:1) (10.5 mg, 0.03 mmol) in toluene (660 μL), imine (18.3 mg, 0.1 mmol) was added at room temperature (25° C.) followed by 3-hepten-2-one (39.6 μL, 0.3 mmol), and the mixture was stirred at the same temperature for 72 h (formation of the product was monitored by ¹H NMR analyses; ¹H NMR yield of the desired product>95%). For the ¹H NMR analysis, a portion of the reaction mixture was taken out and diluted with CDCl₃. The ¹H NMR yield of the product was determined based on the ratio between the remaining imine and the desired product before purification (the uncyclized Mannich product was not detected at 72 h). The mixture was directly purified by flash column chromatography (hexane/EtOAc=70:30) to give the product (Compound 12). The enantiomer ratio (er) of the product was determined by HPLC analysis. HPLC (Daicel Chiralpak IC3, hexane/i-PrOH=90:10, flow rate=0.8 mL/min, λ=220 nm): tR (major enantiomer)=21 min, tR (minor enantiomer)=36 min.

### INDUSTRIAL APPLICABILITY

According to the present disclosure, novel 1,3-diamine derivatives and their applications, including catalytic uses, are provided.

1. A compound represented by a formula (I) or a salt thereof 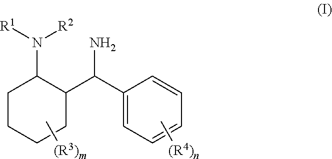
wherein
R¹ and R² are independently selected from the group consisting of an alkyl, an alkenyl, an alkynyl, a heteroalkyl, a heteroalkenyl, or a heteroalkynyl, which may be substituted with one or more selected from the group consisting of an aryl and a heteroaryl, or
R¹ and R² together form —(CH₂)ₚ—, and the —(CH₂)ₚ— forms a ring in combination with the nitrogen atom that the R¹ and the R² are both attached to,
wherein p is an integer from 2 to 8, and
the —(CH₂)ₚ— may be interrupted by one to three hetero atom(s) and may be substituted with one or more selected from the group consisting of an alkyl, an aryl, and a heteroaryl;
R³ is independently selected from the group consisting of an alkyl, and an aryl, or
two adjacent R³s may form a ring with carbon atoms they are attached to;
R⁴ is independently selected from the group consisting of a halogen atom, an alkyl, an alkoxy, a cyano, a nitro, and an acylamido, or
two adjacent R⁴s may form a ring in combination with carbon atoms they are attached to;
m is an integer from 0 to 8; and
n is an integer from 0 to 5.

2. The compound or the salt thereof according to claim 1, wherein the compound is represented by formula (1-1) or (1-2) 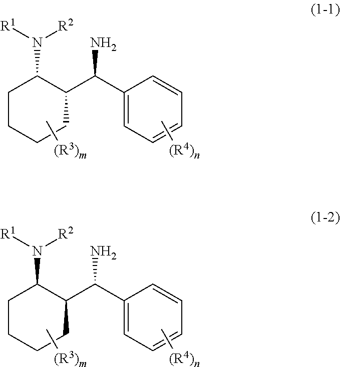
wherein R¹, R², R³, R⁴, m, and n are as defined in claim 1.

3. A catalyst comprising a compound or a salt thereof according to claim 1.

4. The catalyst according to claim 3 wherein it is used in a Mannich reaction.

5. A method of preparing a compound or a salt thereof according to claim 1 comprising:
converting an —NHPMP moiety of a compound represented by a formula (II) to an amino group; and
introducing the amino group by reductive amination of a carbonyl group of the compound represented by the formula (II), followed by converting the introduced amino group to a tertiary amino group to form an —NR¹R² moiety, wherein R¹, and R² are as defined in claim 1 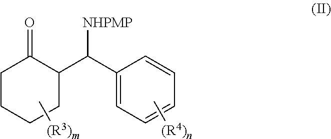
wherein
PMP is a p-methoxyphenyl group, and
R³, R⁴, m, and n are as defined in claim 1.

6. A method of preparing a Mannich product, comprising:
reacting a compound represented by formula (VIII) with a compound represented by formula (VII), in the presence of a compound represented by the formula (I) according to claim 1 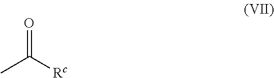
wherein
R^{c} is a hydrogen atom or monovalent carbon atom which may be substituted with a —P(═O)(OR^{d})(OR^{e}), a —C(═O)R^{d}, a —S(═O)₂R^{d}, a —C(═O)OR^{d}, or a —C(═O)NR^{f}R^{g},
wherein
R^{d} is an alkyl,
R^{e} is an alkyl,
R^{f} is a hydrogen atom or an alkyl, and
R^{g} is a hydrogen atom or an alkyl; 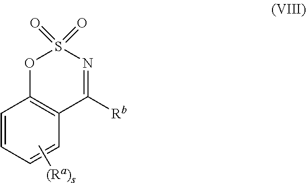
wherein
R^{a} is independently selected from the group consisting of an alkyl, a halogen atom, an alkoxy, and a nitro, or
two adjacent Ras may form a ring in combination with carbon atoms they are attached to;
s is an integer of 0 to 4; and
R^{b} is a hydrogen atom or an alkoxycarbonyl.

7. The method of preparing a Mannich product according to claim 6, comprising:
reacting the compound represented by the formula (VIII) with the compound represented by the formula (VII), in the presence of the compound represented by the formula (1-1) to obtain a compound represented by formula (9-1), or in the presence of the compound represented by the formula (1-2) to obtain a compound represented by formula (9-2) 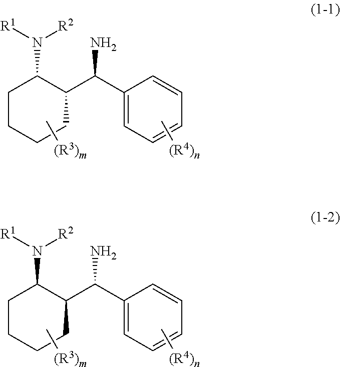
wherein
R¹ and R² are independently selected from the group consisting of an alkyl, an alkenyl, an alkynyl, a heteroalkyl, a heteroalkenyl, or a heteroalkynyl, which may be substituted with one or more selected from the group consisting of an aryl and a heteroaryl, or
R¹ and R² together form —(CH₂)ₚ—, and the —(CH₂)ₚ— forms a ring in combination with the nitrogen atom that the R¹ and the R² are both attached to,
wherein p is an integer from 2 to 8, and
the —(CH₂)ₚ— may be interrupted by one to three hetero atom(s) and may be substituted with one or more selected from the group consisting of an alkyl, an aryl, and a heteroaryl;
R³ is independently selected from the group consisting of an alkyl, and an aryl, or
two adjacent R³s may form a ring with carbon atoms they are attached to:
R⁴ is independently selected from the group consisting of a halogen atom, an alkyl, an alkoxy, a cyano, a nitro, and an acylamido, or
two adjacent R⁴s may form a ring in combination with carbon atoms they are attached to:
m is an integer from 0 to 8; and
n is an integer from 0 to 5, 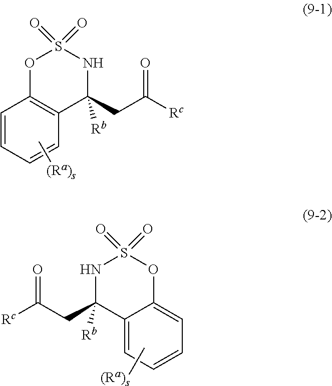
wherein R^{a}, R^{b}, R^{c} and s are as defined in claim 6.

8. The method of preparing a Mannich product according to claim 6, comprising:
reacting the compound represented by the formula (VIII) with the compound represented by the formula (VII′) to obtain a compound represented by formula (9-3) 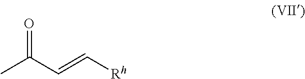
wherein R^{h} is a hydrogen atom, a —P(═O)(OR^{d})(OR^{e}), a —C(═O)R^{d} a —S(═O)₂R^{d}, a —C(═O)OR^{d}, a —C(═O)NR^{f}R^{g}, or a monovalent hydrocarbon group which may be substituted with a —P(═O)(OR^{d})(OR^{e}), a —C(═O)R^{d}, a —S(═O)₂R^{d}, a —C(═O)OR^{d}, or a —C(═O)NR^{f}R^{g},
wherein R^{d}, R^{e}, R^{f}, and R^{g} are as defined in claim 6; 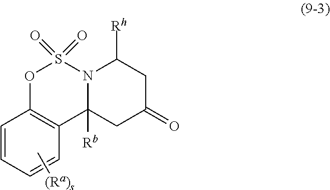
wherein
R^{a}, R^{b}, and s are as defined in claim 6; and
R^{h} is as defined in the above.

9. A catalyst comprising a compound or a salt thereof according to claim 2.

10. The catalyst according to claim 9 wherein it is used in a Mannich reaction.
